SEQUENCE LISTING (104 sequences)

SEQ ID NO: 1 - protein, 289 aa, Bacteriophage A500
MALTEAWLIEKANRKLNAGGMYKITSDKTRNVIKKMAKEGIYLCVAQGYRSTAEQNALYAQGRTKPGAIVTNAKGGQSNHNYGVAVDLCLYTNDGKDVIWESTTSRWKKVVAAMKAEGFKWGGDWKSFKDYPHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 2 - DNA, 870 bp, Bacteriophage A500
atggcattaacagaggcatggctaattgaaaaagcaaatcgcaaattgaatgctgggggaatgtataaaattacatcggataaaacacgaaatgtaattaaaaaaatggcaaaagaaggtatttatctttgtgttgcgcaaggttaccgctcaacagcggaacaaaatgcgctatatgcacaagggagaaccaaacctggagcaattgttactaatgccaagggcgggcaatctaatcacaactacggggtagctgttgacttgtgcttgtatacaaatgacggaaaagatgttatttgggagtcaacaacttcccggtggaaaaaggttgttgctgctatgaaagcagaagggtttaaatggggcggagactggaaaagttttaaagactatccgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 3 - protein, 281 aa, Bacteriophage A118
MTSYYYSRSLANVNKLADNTKAAARKLLDWSESNGIEVLIYETIRTKEQQAANVNSGASQTMRSYHLVGQALDFVMAKGKTVDWGAYRSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 4 - DNA, 846 bp, Bacteriophage A118
atgacaagttattattatagtagaagtttagcgaatgtaaataagttagcagacaatacgaaagcggcacgtagaaaattgctagattggtctgaaagcaacgggattgaagtattaatctacgaaacaattagaacgaaagaacaacaagccgcaaatgttaacagtggagcgtctcaaacaatgcgctcttatcacttggtaggacaagcgctagatttcgtcatggcgaaaggtaaaacggtcgattggggtgcttatcgttcagacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttag

SEQ ID NO: 5 - protein, 291 aa, Bacteriophage P35
MARKFTKAELVAKAEKKVGGLKPDVKKAVLSAVKEAYDRYGIGIIVSQGYRSIAEQNGLYAQGRTKPGNIVTNAKGGQSNHNFGVAVDFAIDLIDDGKIDSWQPSATIVNMMKRRGFKWGGDWKSFTDLPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 6 - DNA, 876 bp, Bacteriophage P35
atggcacgaaaatttacaaaagctgaactggtagctaaagcagaaaagaaagtcggtggattaaaacccgacgtaaagaaagcagtattgtccgcagtgaaggaagcatatgaccgctatggtattgggattatcgtatcacagggttatcgttcaattgctgaacaaaacggattgtatgcacaaggtcggaccaaaccggggaacattgtgaccaacgcaaaaggtggacaatctaaccataactttggtgttgccgttgactttgctattgacttgattgacgatggtaaaatcgactcttggcaaccatcagcaaccattgtgaacatgatgaaacgtcgtgggttcaaatggggcggagattggaaaagctttactgaccttccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaatga

SEQ ID NO: 7 - protein, 363 aa, artificial sequence
  CBD500-118
ITHGMDELYKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 8 - DNA, 1092 bp, artificial sequence
  CBD500-118
attacacatggcatggatgaactatacaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 9 - protein, 348 aa, artificial sequence
  CBD500L118
ITHGMDELYKELQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKTGKTVAAKNPNRHSKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 10 - DNA, 1047 bp, artificial sequence
  CBD500L118
attacacatggcatggatgaactatacaaagagctccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaaactggtaaaacagtagccgcaaaaaatccaaaccgccattctaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 11 - protein, 14 aa, artificial sequence
  PlyPSA linker (L)
TGKTVAAKNPNRHS

SEQ ID NO: 12 - DNA, 42 bp, artificial sequence
  PlyPSA linker (L)
actggtaaaacagtagccgcaaaaaatccaaaccgccattct

SEQ ID NO: 13 - protein, 363 aa, artificial sequence
  CBD118-500
ITHGMDELYKELDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKIELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 14 - DNA, 1092 bp, artificial sequence
  CBD118-500
attacacatggcatggatgaactatacaaagagctcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattgaattgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 15 - protein, 348 aa, artificial sequence
  CBD118L500
ITHGMDELYKELKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKITGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 16 - DNA, 1047 bp, artificial sequence
  CBD118L500
attacacatggcatggatgaactatacaaagagctcaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 17 - protein, 316 aa, artificial sequence
  CBD500-P35
ITHGMDELYKELQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 18 - DNA, 951 bp, artificial sequence
  CBD500-P35
attacacatggcatggatgaactatacaaagagctccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaataa

SEQ ID NO: 19 - protein, 316 aa, artificial sequence
  CBDP35-500
ITHGMDELYKELPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKKQFQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 20 - DNA, 951 bp, artificial sequence
  CBDP35-500
attacacatggcatggatgaactatacaaagagctcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaacaattccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 21 - protein, 311 aa, artificial sequence
  CBD500-500
ITHGMDELYKELQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 22 - DNA, 936 bp, artificial sequence
  CBD500-500
attacacatggcatggatgaactatacaaagagctccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 23 - protein, 450 aa, artificial sequence
  EAD_CBD500-500
GSMALTEAWLIEKANRKLNAGGMYKITSDKTRNVIKKMAKEGIYLCVAQGYRSTAEQNALYAQGRTKPGAIVTNAKGGQSNHNYGVAVDLCLYTNDGKDVIWESTTSRWKKVVAAMKAEGFKWGGDWKSFKDYPHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 24 - DNA, 1353 bp, artificial sequence
  EAD_CBD500-500
ggatccatggcattaacagaggcatggctaattgaaaaagcaaatcgcaaattgaatgctgggggaatgtataaaattacatcggataaaacacgaaatgtaattaaaaaaatggcaaaagaaggtatttatctttgtgttgcgcaaggttaccgctcaacagcggaacaaaatgcgctatatgcacaagggagaaccaaacctggagcaattgttactaatgccaagggcgggcaatctaatcacaactacggggtagctgttgacttgtgcttgtatacaaatgacggaaaagatgttatttgggagtcaacaacttcccggtggaaaaaggttgttgctgctatgaaagcagaagggtttaaatggggcggagactggaaaagttttaaagactatccgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 25 - protein, 317 aa, artificial sequence
  Fab25VL
MAYKVERRIRPGLPQVGYAPYGQVHAHSTGNPRSTAQNEADYFQTKDITTGFYTHLVGNGRVIQLAEVNRGAWDVGGGWNQWGYASVELIESHSNRDEFMRDYKIYCELLHDLAKQAGLPTTVDQGNTGIITHNYATHNQPNNGSDHVDPIPYLAKWGINLAQFRSDVANAKSNSKPVTPSKPVSHDKAIAKSPAKTVNGYTGKMDKFNVQGNKFRVAGWMLPTAGGQPYNYGYVFLLDAKTGKEIARQLAGAVSRPDVTKAYGVKGGTNYGLDVTFDVKKLKGKKFYAMFRRTNDKAGNTAGGHKDIGFNEFYFTL

SEQ ID NO: 26 - DNA, 954 bp, artificial sequence
  Fab25VL
atggcttataaagtagaaagacgaattagaccagggttgccccaagttgggtatgctccatatggacaagtacacgcacacagtacaggtaacccacgtagtacagcacaaaatgaggcagattattttcagactaaagacattaccactggtttttacactcatcttgtaggtaatggacgagtaatccaactagctgaagttaatcgtggcgcatgggatgttggtggaggttggaatcaatggggctatgcatcagttgaattaattgaatcccactctaatagagacgagtttatgcgtgattataagatttattgtgagttactgcatgacttagcaaaacaagcaggcttacctaccactgttgaccaaggtaacactggtatcatcactcataactatgcaacacataatcaaccaaacaatggttctgaccatgttgaccctattccatatcttgctaaatggggtatcaatttagctcagtttagaagtgatgttgctaacgctaagagtaacagcaagcctgtgacaccaagcaaaccggtgtcccatgataaagctatcgctaaaagtcctgctaaaacagttaatggatatactggtaaaatggataagttcaatgttcaaggtaataagtttcgtgtggcaggatggatgttacctaccgcaggcggtcaaccttacaactatggttacgtatttttacttgatgctaaaacaggtaaagaaattgcacgtcaacttgcaggtgcggtgtctcgtcctgatgtaactaaagcatatggcgtaaaaggtggcactaactatggtctagatgttacctttgatgttaaaaaactaaaaggtaaaaaattctatgcaatgtttagacgtaccaatgataaagcaggtaatactgctggtggacataaagacattgggttcaatgaattctacttcacgctataa

SEQ ID NO: 27 - protein, 365 aa, artificial sequence
  Fab20VL
MKLKGILFGALATIGLFAGMQTANAYEVNNEFNLSPWEGSGQVAVPNKIVLHETANERATGRNEATYMKNNWFNAHTTAIIGDGGIVYKIAPEGNISWGAGNANPYAPIQIELQHTHDKELFKKNYKAYIDYTRDMGKKFGIPMTLDQGSSVWEKGVISHKWVSDYVWGDHTDPYGYLAEMGISKAQLTKDLANGVSGESVKPTPSKPKTFKKGQNVYIYNGHKSHNGPVVPFVAGSSLWTQVGTITEVKQGSVNPYKIENSGKFVTYANAGDLEDLNTKFPPKPSKPVSQFTIGVDAIVLRSGRPSVYAPVYGTWKQGAVFKYDEITVGDGYVWIGGTDTNGTRIYLPIGPNDGDPNNTWGTLV

SEQ ID NO: 28 - DNA, 1098 bp, artificial sequence
  Fab20VL
atgaagttaaaaggtattttatttggtgcattagcaaccattggtttgtttgctggaatgcaaacagctaatgcatatgaagttaataacgagttcaatttaagtccttgggaaggttcaggacaggttgcagtacctaataagattgtcttacatgaaactgctaatgaacgtgccacaggacggaatgaagcaacgtacatgaaaaacaactggtttaatgctcatacaacagctatcattggtgatggtggtattgtttataagattgcaccagaaggtaacatttcatggggtgctggtaatgcaaacccctacgcacctattcaaattgagttacaacatacacatgataaagagttgttcaaaaagaactataaagcgtacattgactatacaagagacatgggtaaaaagtttggtattcctatgacgcttgaccaaggttcttctgtttgggaaaaaggtgttatctctcataaatgggtatcagattatgtatggggtgaccacacagacccatatggttacttagcagaaatgggaatcagtaaagcacaacttactaaagacttagccaatggggtatctggtgaatcagtaaaaccaacaccaagcaaaccaaagacattcaaaaaaggtcaaaatgtttacatttataacggtcacaagtcacacaatggacctgtagtaccatttgtagctggctcaagtctttggacccaagttggtacaattacagaagtgaaacaaggttcagtcaatccgtataagattgaaaacagtggtaaatttgtaacatatgctaacgctggcgacttagaggaccttaacactaagttcccaccaaaaccaagcaaaccagttagtcagtttacaattggtgttgacgctattgttttacgtagtggacgacctagcgtatatgcaccagtatacggaacatggaaacaaggtgcagtattcaagtatgatgaaatcacagttggtgatggctatgtatggattggtggaacagacactaatggtacacgtatttacttaccaattggaccaaatgacggagaccctaacaacacgtggggtacattagtataa

SEQ ID NO: 29 - protein, 346 aa, artificial sequence
  Fab20K
MQTANAYEVNNEFNLSPWEGSGQVAVPNKIVLHETANERATGRNEATYMKNNWFNAHTTAIIGDGGIVYKIAPEGNISWGAGNANPYAPIQIELQHTHDKELFKKNYKAYIDYTRDMGKKFGIPMTLDQGSSVWEKGVISHKWVSDYVWGDHTDPYGYLAEMGISKAQLTKDLANGVSGESVKPTPSKPKTFKKGQNVYIYNGHKSHNGPVVPFVAGSSLWTQVGTITEVKQGSVNPYKIENSGKFVTYANAGDLEDLNTKFPPKPSKPVSQFTIGVDAIVLRSGRPSVYAPVYGTWKQGAVFKYDEITVGDGYVWIGGTDTNGTRIYLPIGPNDGDPNNTWGTLV

SEQ ID NO: 30 - DNA, 1041 bp, artificial sequence
  Fab20K
atgcaaacagctaatgcatatgaagttaataacgagttcaatttaagtccttgggaaggttcaggacaggttgcagtacctaataagattgtcttacatgaaactgctaatgaacgtgccacaggacggaatgaagcaacgtacatgaaaaacaactggtttaatgctcatacaacagctatcattggtgatggtggtattgtttataagattgcaccagaaggtaacatttcatggggtgctggtaatgcaaacccctacgcacctattcaaattgagttacaacatacacatgataaagagttgttcaaaaagaactataaagcgtacattgactatacaagagacatgggtaaaaagtttggtattcctatgacgcttgaccaaggttcttctgtttgggaaaaaggtgttatctctcataaatgggtatcagattatgtatggggtgaccacacagacccatatggttacttagcagaaatgggaatcagtaaagcacaacttactaaagacttagccaatggggtatctggtgaatcagtaaaaccaacaccaagcaaaccaaagacattcaaaaaaggtcaaaatgtttacatttataacggtcacaagtcacacaatggacctgtagtaccatttgtagctggctcaagtctttggacccaagttggtacaattacagaagtgaaacaaggttcagtcaatccgtataagattgaaaacagtggtaaatttgtaacatatgctaacgctggcgacttagaggaccttaacactaagttcccaccaaaaccaagcaaaccagttagtcagtttacaattggtgttgacgctattgttttacgtagtggacgacctagcgtatatgcaccagtatacggaacatggaaacaaggtgcagtattcaagtatgatgaaatcacagttggtgatggctatgtatggattggtggaacagacactaatggtacacgtatttacttaccaattggaccaaatgacggagaccctaacaacacgtggggtacattagtataa

SEQ ID NO: 31 - protein, 485 aa, artificial sequence
  EAD_Fab25_CBD25_CBD20
MAYKVERRIRPGLPQVGYAPYGQVHAHSTGNPRSTAQNEADYFQTKDITTGFYTHLVGNGRVIQLAEVNRGAWDVGGGWNQWGYASVELIESHSNRDEFMRDYKIYCELLHDLAKQAGLPTTVDQGNTGIITHNYATHNQPNNGSDHVDPIPYLAKWGINLAQFRSDVANAKSNSKPVTPSKPVSHDKAIAKSPAKTVNGYTGKMDKFNVQGNKFRVAGWMLPTAGGQPYNYGYVFLLDAKTGKEIARQLAGAVSRPDVTKAYGVKGGTNYGLDVTFDVKKLKGKKFYAMFRRTNDKAGNTAGGHKDIGFNEFYFTLELMVKPTPSKPKTFKKGQNVYIYNGHKSHNGPVVPFVAGSSLWTQVGTITEVKQGSVNPYKIENSGKFVTYANAGDLEDLNTKFPPKPSKPVSQFTIGVDAIVLRSGRPSVYAPVYGTWKQGAVFKYDEITVGDGYVWIGGTDTNGTRIYLPIGPNDGDPNNTWGTLV

SEQ ID NO: 32 - DNA, 1458 bp, artificial sequence
  EAD_Fab25_CBD25_CBD20
atggcttataaagtagaaagacgaattagaccagggttgccccaagttgggtatgctccatatggacaagtacacgcacacagtacaggtaacccacgtagtacagcacaaaatgaggcagattattttcagactaaagacattaccactggtttttacactcatcttgtaggtaatggacgagtaatccaactagctgaagttaatcgtggcgcatgggatgttggtggaggttggaatcaatggggctatgcatcagttgaattaattgaatcccactctaatagagacgagtttatgcgtgattataagatttattgtgagttactgcatgacttagcaaaacaagcaggcttacctaccactgttgaccaaggtaacactggtatcatcactcataactatgcaacacataatcaaccaaacaatggttctgaccatgttgaccctattccatatcttgctaaatggggtatcaatttagctcagtttagaagtgatgttgctaacgctaagagtaacagcaagcctgtgacaccaagcaaaccggtgtcccatgataaagctatcgctaaaagtcctgctaaaacagttaatggatatactggtaaaatggataagttcaatgttcaaggtaataagtttcgtgtggcaggatggatgttacctaccgcaggcggtcaaccttacaactatggttacgtatttttacttgatgctaaaacaggtaaagaaattgcacgtcaacttgcaggtgcggtgtctcgtcctgatgtaactaaagcatatggcgtaaaaggtggcactaactatggtctagatgttacctttgatgttaaaaaactaaaaggtaaaaaattctatgcaatgtttagacgtaccaatgataaagcaggtaatactgctggtggacataaagacattgggttcaatgaattctacttcacgctagagctcatggtaaaaccaacaccaagcaaaccaaagacattcaaaaaaggtcaaaatgtttacatttataacggtcacaagtcacacaatggacctgtagtaccatttgtagctggctcaagtctttggacccaagttggtacaattacagaagtgaaacaaggttcagtcaatccgtataagattgaaaacagtggtaaatttgtaacatatgctaacgctggcgacttagaggaccttaacactaagttcccaccaaaaccaagcaaaccagttagtcagtttacaattggtgttgacgctattgttttacgtagtggacgacctagcgtatatgcaccagtatacggaacatggaaacaaggtgcagtattcaagtatgatgaaatcacagttggtgatggctatgtatggattggtggaacagacactaatggtacacgtatttacttaccaattggaccaaatgacggagaccctaacaacacgtggggtacattagtataa

SEQ ID NO: 33 - protein, 603 aa, artificial sequence
  HGFP_CBD118-500
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKIELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 34 - DNA, 1812 bp, artificial sequence
  HGFP_CBD118-500
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattgaattgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 35 - protein, 603 aa, artificial sequence
  HGFP_CBD500-118
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 36 - DNA, 1812 bp, artificial sequence
  HGFP_CBD500-118
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 37 - protein, 588 aa, artificial sequence
  HGFP_CBD118L500
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKITGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 38 - DNA, 1767 bp, artificial sequence
  HGFP_CBD118L500
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctcaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 39 - protein, 588 aa, artificial sequence
  HGFP_CBD500L118
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKTGKTVAAKNPNRHSKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 40 - DNA, 1767 bp, artificial sequence
  HGFP_CBD500L118
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaaactggtaaaacagtagccgcaaaaaatccaaaccgccattctaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 41 - protein, 556 aa, artificial sequence
  HGFP_CBD500-P35
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 42 - DNA, 1671 bp, artificial sequence
  HGFP_CBD500-P35
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaataa

SEQ ID NO: 43 - protein, 556 aa, artificial sequence
  HGFP_CBDP35-500
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKKQFQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 44 - DNA, 1671 bp, artificial sequence
  HGFP_CBDP35-500
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaacaattccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 45 - protein, 586 aa, artificial sequence
  HCBD500_GFP_CBD118
MRGSHHHHHHGSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKGTMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 46 - DNA, 1761 bp, artificial sequence
  HCBD500_GFP_CBD118
atgagaggatcgcatcaccatcaccatcacggatcccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaaggtaccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 47 - protein, 601 aa, artificial sequence
  HCBD118_GFP_CBD500
MRGSHHHHHHGSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKIMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 48 - DNA, 1806 bp, artificial sequence
  HCBD118_GFP_CBD500
atgagaggatcgcatcaccatcaccatcacggatccgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 49 - protein, 551 aa, artificial sequence
  HGFP_CBD500-500
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 50 - DNA, 1656 bp, artificial sequence
  HGFP_CBD500-500
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagagctccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 51 - protein, 460 aa, artificial sequence
  HEAD_CBD500-500
MRGSHHHHHHGSMALTEAWLIEKANRKLNAGGMYKITSDKTRNVIKKMAKEGIYLCVAQGYRSTAEQNALYAQGRTKPGAIVTNAKGGQSNHNYGVAVDLCLYTNDGKDVIWESTTSRWKKVVAAMKAEGFKWGGDWKSFKDYPHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 52 - DNA, 1383 bp, artificial sequence
  HEAD_CBD500-500
atgagaggatcgcatcaccatcaccatcacggatccatggcattaacagaggcatggctaattgaaaaagcaaatcgcaaattgaatgctgggggaatgtataaaattacatcggataaaacacgaaatgtaattaaaaaaatggcaaaagaaggtatttatctttgtgttgcgcaaggttaccgctcaacagcggaacaaaatgcgctatatgcacaagggagaaccaaacctggagcaattgttactaatgccaagggcgggcaatctaatcacaactacggggtagctgttgacttgtgcttgtatacaaatgacggaaaagatgttatttgggagtcaacaacttcccggtggaaaaaggttgttgctgctatgaaagcagaagggtttaaatggggcggagactggaaaagttttaaagactatccgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 53 - protein, 10 aa, artificial sequence
  His Tag
MRGSHHHHHH

SEQ ID NO: 54 - DNA, 30 bp, artificial sequence
  His Tag
atgagaggatcgcatcaccatcaccatcac

SEQ ID NO: 55 - protein, 228 aa, artificial sequence
  GFP
MSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAG

SEQ ID NO: 56 - DNA, 684 bp, artificial sequence
  GFP
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctggg

SEQ ID NO: 57 - DNA, 615 bp, artificial sequence
  CBD118
attacacatggcatggatgaactatacaaagagctcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 58 - DNA, 510 bp, artificial sequence
  CBD500
attacacatggcatggatgaactatacaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 59 - DNA, 525 bp, artificial sequence
  CBDP35
attacacatggcatggatgaactatacaaagagctcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaataa

SEQ ID NO: 60 - protein, 409 aa, artificial sequence
  HGFP_CBD500
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 61 - protein, 6 aa, artificial sequence
  PlyPSA linker
AAKNPN

SEQ ID NO: 62 - DNA, 18 bp, artificial sequence
  PlyPSA linker
gccgcaaaaaatccaaac

SEQ ID NO: 63 - protein, 9 aa, artificial sequence
  Polypeptidelinker
AGAGAGAGS

SEQ ID NO: 64 - protein, 11 aa, artificial sequence
  Polypeptidelinker
AGAGAGAGSEL

SEQ ID NO: 65 - protein, 14 aa, artificial sequence
  Polypeptidelinker
TPTPPNPGPKNFTT

SEQ ID NO: 66 - protein, 9 aa, artificial sequence
  Polypeptidelinker
AGAGAGAGL

SEQ ID NO: 67 - DNA, 1341 bp, artificial sequence
  HGFP_L_CBD118 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 68 - protein, 446 aa, artificial sequence
  HGFP_L_CBD118 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 69 - DNA, 1251 bp, artificial sequence
  HGFP_ L_CBDP35 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaataa

SEQ ID NO: 70 - protein, 416 aa, artificial sequence
  HGFP_ L_CBDP35 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 71 - protein, 605 aa, artificial sequence
  HGFP_ L_CBD500-118 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 72 - DNA, 1818 bp, artificial sequence
  HGFP_ L_CBD500-118 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 73 - protein, 352 aa, artificial sequence
  CBD500-118
MHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 74 - DNA, 1059 bp, artificial sequence
  CBD500-118
atgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 75 - protein, 605 aa, artificial sequence
  HGFP_ L_CBD118-500 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKIELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 76 - DNA, 1818 bp, artificial sequence
  HGFP_ L_CBD118-500 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattgaattgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 77 - protein, 352 aa, artificial sequence
  CBD118-500
MDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKIELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 78 - DNA, 1059 bp, artificial sequence
  CBD118-500
atggacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattgaattgcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 79 - protein, 590 aa, artificial sequence
  HGFP_ L_CBD500L118 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKTGKTVAAKNPNRHSKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 80 - DNA, 1773 bp, artificial sequence
  HGFP_ L_CBD500L118 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaaactggtaaaacagtagccgcaaaaaatccaaaccgccattctaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 81 - protein, 337 aa, artificial sequence
  CBD500L118
MQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKTGKTVAAKNPNRHSKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 82 - DNA, 1014 bp, artificial sequence
  CBD500L118
atgcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaaactggtaaaacagtagccgcaaaaaatccaaaccgccattctaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 83 - protein, 590 aa, artificial sequence
  HGFP_ L_CBD118L500 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKITGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 84 - DNA, 1773 bp, artificial sequence
  HGFP_ L_CBD118L500 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 85 - protein, 337 aa, artificial sequence
  CBD118L500
MKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKITGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 86 - DNA, 1014 bp, artificial sequence
  CBD118L500
atgaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 87 - protein, 588 aa, artificial sequence
  HCBD500_GFP_ L_118 (mit PlyPSA linker)
MRGSHHHHHHGSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKGTMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKI

SEQ ID NO: 88 - DNA, 1767 bp, artificial sequence
  HCBD500_GFP_ L_118 (mit PlyPSA linker)
atgagaggatcgcatcaccatcaccatcacggatcccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaaggtaccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagatttaa

SEQ ID NO: 89 - protein, 603 aa, artificial sequence
  HCBD118_GFP_ L_500 (mit PlyPSA linker)
MRGSHHHHHHGSDKGKKFVAKAKSLGFEWGGDWSGFVDNPHLQFNYKGYGTDTFGKGASTSNSSKPSADTNTNSLGLVDYMNLNKLDSSFANRKKLATSYGIKNYSGTATQNTTLLAKLKAGKPHTPASKNTYYTENPRKVKTLVQCDLYKSVDFTTKNQTGGTFPPGTVFTISGMGKTKGGTPRLKTKSGYYLTANTKFVKKIMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 90 - DNA, 1812 bp, artificial sequence
  HCBD118_GFP_ L_500 (mit PlyPSA linker)
atgagaggatcgcatcaccatcaccatcacggatccgacaaaggcaagaaatttgtggcaaaggcaaaatctttaggttttgagtggggtggtgattggtctggatttgtagacaatccgcaccttcaatttaattataaaggctatgggactgatacttttggaaaaggagctagtactagtaattcatctaaaccgagcgcagacacaaacacaaacagtctaggattagtagattatatgaatttaaataaactagattcaagctttgcgaatcgcaaaaaactagcgacaagttacggaattaaaaattacagtggaacagcaacgcagaacacaacattattagcgaagttaaaagcaggaaaaccacacacaccagcaagcaaaaacacatactacacagaaaatccgcgaaaagttaaaacactagtacaatgtgatctatacaaatcagtagactttacaacaaaaaaccaaacaggtggaacatttccgccaggcacagtcttcacgatttcagggatggggaaaacgaaaggcggaacacctcgcttgaagacgaagagcggttactatctcactgctaacacgaagtttgttaaaaagattatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctcattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 91 - protein, 558 aa, artificial sequence
  HGFP_ L_CBD500-P35 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 92 - DNA, 1677 bp, artificial sequence
  HGFP_ L_CBD500-P35 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaataa

SEQ ID NO: 93 - protein, 305 aa, artificial sequence
  CBD500-P35
MQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKQFPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKK

SEQ ID NO: 94 - DNA, 918 bp, artificial sequence
  CBD500-P35
atgcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaacaattcccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaataa

SEQ ID NO: 95 - protein, 558 aa, artificial sequence
  HGFP_ L_CBDP35-500 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKKQFQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 96 - DNA, 1677 bp, artificial sequence
  HGFP_ L_CBDP35-500 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaacaattccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 97 - protein, 305 aa, artificial sequence
  CBDP35-500
MPHFEACDWYRGERKYKVDTSEWKKKENINIVIKDVGYFQDKPQFLNSKSVRQWKHGTKVKLTKHNSHWYTGVVKDGNKSVRGYIYHSMAKVTSKNSDGSVNATINAHAFCWDNKKLNGGDFINLKRGFKGITHPASDGFYPLYFASRKKTFYIPRYMFDIKKQFQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 98 - DNA, 918 bp, artificial sequence
  CBDP35-500
atgccacattttgaagcttgtgactggtatcgcggggaacgcaagtataaagtggacacatctgaatggaaaaagaaagagaatatcaatatcgttattaaagatgttggttacttccaagacaaacctcaattcttaaactccaaatcggttcgtcagtggaagcatggcacgaaagtgaagcttactaaacataactcacattggtacactggtgtggtcaaggatggtaacaaatcagtcaggggatatatttatcattcgatggctaaggtcacaagcaagaatagcgacggttcggttaacgcaacgattaacgcccacgcattttgttgggacaataaaaaacttaatggtggcgactttatcaacttgaagcgtggttttaaaggtatcacccatcccgctagtgacggtttctatccactgtatttcgcttctaggaaaaaaactttctacattccgcgttacatgtttgacatcaagaaacaattccaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 99 - protein, 553 aa, artificial sequence
  HGFP_ L_CBD500-500 (mit PlyPSA linker)
MRGSHHHHHHGSMSKGEELFTGVVPILVELDGDVNGHKFSVSGEGEGDATYGKLTLKFICTTGKLPVPWPTLVTTFAYGLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGTGKTVAAKNPNRHSQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 100 - DNA, 1662 bp, artificial sequence
  HGFP_ L_CBD500-500 (mit PlyPSA linker)
atgagaggatcgcatcaccatcaccatcacggatccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggactggtaaaacagtagccgcaaaaaatccaaaccgccattctcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 101 - protein, 300 aa, artificial sequence
  CBD500-500
MQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLKELHFELCDAVSGEKIPAATQNTNTNSNRYEGKVIDSAPLLPKMDFKSSPFRMYKVGTEFLVYDHNQYWYKTYIDDKLYYMYKSFCDVVAKKDAKGRIKVRIKSAKDLRIPVWNNIKLNSGKIKWYAPNVKLAWYNYRRGYLELWYPNDGWYYTAEYFLK

SEQ ID NO: 102 - DNA, 903 bp, artificial sequence
  CBD500-500
atgcaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaagagctccattttgaactatgtgatgctgtaagtggtgagaaaatccctgctgcaacacaaaacactaatacaaattcaaatcgttacgagggtaaagtcattgatagcgcaccactgctaccgaaaatggactttaaatcatcaccattccgcatgtataaggtaggaactgagttcttagtatatgatcataatcaatattggtacaagacatacattgatgacaaactttactacatgtataaaagcttttgcgatgttgtagctaaaaaagacgcaaaaggtcgcatcaaagttcgaattaaaagcgcgaaagacttgcgtattccagtctggaataacataaaattgaattctgggaaaattaaatggtatgcacccaatgtaaaactagcgtggtacaactatcgaagaggatatttagagctatggtatccgaacgacggctggtattacacagcagaatacttcttaaaataa

SEQ ID NO: 103 - protein, 200 aa, Bacteriophage P40
MVLVLDISKWQPTVNYSGLKEDVGFVVIRSSNGTQKYDERLEQHAKGLDKVGMPFGLYHYALFEGGQDTINEANMLVSAYKKCRQLGAEPTFLFLDYEEVKLKSGNVVNECQRFIDHVKGQTGVKVGLYAGDSFWKTHDLDKVKHDLRWVARYGVDNGKPSTKPSIPYDLWQYTSKGRIKAIASPVDMNTCSSDILNKLK

SEQ ID NO: 104 - protein, 118 aa, Bacteriophage P40
TTKYVNTAHLNIREKASADSKVLGVLDLNDSVQVISESGGWSKLKSGNKQVYVSSKYLSKSKTTPKAKPSSKQYYTIKSGDNLSYIAKKYKTTVKQIQNWNGIKDANKIYAGQKIRVK

(19) United States
(12) Patent Application Publication
(10) Pub. No.: US 2012/0171188 A1
(43) Pub. Date: Jul. 5, 2012

(54) Artificial Peptidoglycan Lysing Enzymes and Peptidoglycan Binding Proteins

(75) Inventors: Martin Loessner, Ebmatingen (CH); Mathias Schmelcher, Schwabhausen (DE); Holger Grallert, Weilheim (DE); Falko Bretfeld, Regensburg (DE)

(73) Assignees: HYGLOS INVEST GMBH, Bernried (DE); BIOMERIEUX S.A, Marcy L'Etoile (FR)

(21) Appl. No.: 13/251,896

(22) Filed: Oct. 3, 2011

Related U.S. Application Data
(63) Continuation of application No. 13/059,956, now abandoned.

(30) Foreign Application Priority Data
Aug. 19, 2008 (EP) 08162644.2

(51) Int. Cl. A01N 63/00; A01P 1/00; C12N 15/63; G01N 33/53; C12N 9/96; C07H 21/04

(52) U.S. Cl. 424/94.3; 435/188; 536/23.2; 435/320.1; 435/7.1

(57) ABSTRACT

The present invention relates to recombinant polypeptides having the activity of binding and lysing of bacteria, comprising at least one enzymatically active domain and at least two bacterial cell binding domains. The present invention further relates to recombinant polypeptide having the activity of binding bacteria, comprising at least two bacterial cell binding domain. Further the present inventions relates to nucleic acid molecules comprising a nucleotide sequence encoding the recombinant polypeptides, vectors and host cells.

This application is a continuation of application Serial No. 13/059,956, filed Feb. 18, 2011, now abandoned, which claims priority to PCT/EP2009/060716 filed Aug. 19, 2009 which claims priority to European Patent Application No. EP 08162644.2 filed Aug. 19, 2008. The entire text of each of the above-referenced disclosures are specifically incorporated herein by reference without disclaimer.

The present invention relates to recombinant polypeptides having the activity of binding and lysing of bacteria, comprising at least one enzymatically active domain and at least two bacterial cell binding domains. The present invention further relates to recombinant polypeptides having the activity of binding bacteria, comprising at least two bacterial cell binding domains. Further the present inventions relates to nucleic acid molecules comprising a nucleotide sequence encoding the recombinant polypeptides, vectors and host cells.

In recent years, peptidoglycan-degrading enzymes like bacteriophage endolysins have received increasing attention as antimicrobial agents. In view of emerging and spreading resistance of pathogenic bacteria against classical antibiotics, the demand for alternative ways of controlling these organisms is rising. Especially in case of Gram-positive bacteria, the application of phage endolysins as so called enzybiotics is a promising approach. Due to the absence of an outer membrane in Gram-positives, these enzymes also work as exolysins, i.e. they can cause lysis of susceptible cells from without. This property can be exploited e.g. in molecular biology for the efficient recovery of nucleic acids and proteins from bacterial cells, as it was demonstrated for endolysins from phages infecting Listeria. Aiming towards an application for control of the foodborne pathogen Listeria monocytogenes, genes coding for these lysins were introduced into a number of lactic acid bacteria including Lactococcus lactis and several lactobacilli, which are used as starter organisms in cheese production. Overexpressing and secreting the endolysins, these bacteria consequently showed lytic activity against L. monocytogenes cells. Medical applications of phage encoded peptidoglycan hydrolases reported so far include the detection and killing of Bacillus anthracis, and the control of Streptococcus pneumoniae in vitro and in mouse models. Endolysins are cell wall lytic enzymes which are encoded in the late gene region of dsDNA phages and produced at the end of the lytic multiplication cycle. The same enzymes are also found within prophage genomes integrated into bacterial genomes. Their function is degradation of the bacterial peptidoglycan from within, resulting in lysis of the host cell and release of the phage progeny. According to their different target bonds within the peptidoglycan, endolysins can be divided into 5 different classes: (i) N-acetyl-β-D-muramidases (also known as lysozymes) and (ii) N-acetyl-β-D-glucosaminidases, which are both glycosidases and cleave one of the two β-1,4 glycosidic bonds of the glycan strands each; (iii) lytic transglycosylases, which cleave the same bond as muramidases, but by a different mechanism; (iv) N-acetylmuramoyl-L-alanine amidases, which cut between the glycan and the peptide moieties; and (v) endopeptidases, which cleave within the peptide moiety. All endolysins except for lytic transglycosylases are hydrolases. The same enzymatic activities are also found in bacterial enzymes which lyse cell walls of its own or closely related bacteria, the so-called autolysins, and other bacterial cell wall lysing polypeptides like the bacteriocins. Bacterial autolysins are cell wall lytic enzymes, which play important roles in cell wall remodelling, cell division, transformation, or as virulence factors. Together they can be summarized as peptidoglycan lysing enzymes. The enzymatic degradation of the peptidoglycan due to the action of peptidoglycan lysing enzymes results in loss of integrity of the cell wall and finally in cell disruption caused by the high internal pressure.

The feature of killing bacterial cells makes the peptidoglycan lysing enzymes interesting candidates for a use as a prophylactic or therapeutic agent against bacterial infections in humans and animals, as an antimicrobial or enzybiotic for use as a disinfectant in medical, public or private environment, for use as a decontaminant of bacterial contamination in food industry, animal feed or cosmetic industry or as a general surfactant against bacterially contaminated surfaces. Peptidoglycan lysing enzymes are as well useful in bacterial diagnostics as they specifically bind and lyse bacterial cells from distinct bacterial groups, genera, species, strains or serovars. Specific cell lysis is often combined with additional detection methods relying on the cellular content of the bacterial cells to be detected like nucleic acid based methods or immunological methods.

Endolysins, autolysins and other peptidoglycan lysing enzymes from a Gram-positive background show a modular organization in which catalytic activity and substrate recognition are separated and localized in at least two distinct functional domains, the enzymatically active domain (EAD) and the cell binding domain (CBD). Most endolysins from phages infecting Gram-negative hosts are single-domain globular proteins. However, recently two lysins from a Gram-negative background that consist of two functional domains were reported: The endolysins of the Pseudomonas aeruginosa phages ΦKZ and EL consist of an N-terminal cell binding domain and a C-terminal catalytic domain (Briers et al. Molecular Microbiology, Volume 65, Number 5, September 2007, pp. 1334-1344(11). In contrast, the majority of endolysins from phages infecting Gram-positive bacteria feature a reverse orientation of the domains, with an N-terminal EAD and a C-terminal CBD.

For only very few endolysins, the ligands in the bacterial cell wall recognized by the binding domain are known. The pneumococcal phage Cpl-1 lysozyme specifically recognizes choline containing teichoic acids in the cell wall of Streptococcus pneumoniae, which places it in the family of Choline-Binding Proteins (CBP). The cholin binding modules (CBM) of these proteins are formed by a repeat of about 20 amino acid residues found in multiple tandem copies, ranging from 4 to 18. Cpl-1 exemplifies the modular design of these enzymes with two separate domains—an EAD and a CBD—usually connected via a short linker region. Although the majority of modular phage endolysins consist of one catalytic and one substrate binding domain, there are a number of proteins that harbor two different enzymatic activities, as e.g. the endolysins of Streptococcus agalactiae phage B30 (muramidase and endopeptidase), Staphylococcus aureus phage Φ11 (endopeptidase and amidase), Streptococcus agalactiae phage NCTC 11261 (endopeptidase and muramidase), and Staphylococcus warneri M phage ΦWMY (endopeptidase and amidase).

The fact that peptidoglycan lysing enzymes with phage and bacterial origin, endolysins as well as autolysins or bacteriocins show similar modular architectures, and that high homologies between distinct domains of bacterial and phage derived lytic proteins can be found, suggests a common ancestry and co-evolution of these proteins by interchange of functional domains (Garcia et al., 1990, Gene 86, 81-88). Diaz et al. (1990, Proc. Natl. Acad. Sci., 87, 8125-8129) created chimaeras of phage and bacterial pneumococcal enzymes which exhibited combined biochemical properties. Recombinant chimaeras from genes lacking nucleotide homology were constructed in Diaz et al. (1991, J. Biol. Chem., 266, 5464-6571), confirming also the function of the CBDs in substrate recognition. Croux et al. (1993, Mol. Microbiol., 9, 1019-1025) even created chimaeras based on pneumococcal and clostridial cell wall lytic enzymes which led to the switch in enzymatic activity of endolysins towards cells from other bacterial families. Sanz et al. (1996, Eur. J. Biochem., 235, 601-605) constructed multifunctional pneumococcal murein hydrolases by module assembly which comprised two EADs and one CBD. Recently, fusion proteins consisting of lysostaphin, a peptidoglycan hydrolase from Staphylococcus simulans, and the Streptococcus agalactiae phage B30 endolysin, as well as a C-terminally truncated version thereof, were reported (Donovan et al. 2006, Appl. Environ. Microbiol., 72, 2988-2996). Also in this case, the artificial constructs combined properties of both enzymes, lysing both Staphylococcus and Streptococcus cells. Loessner et al. (2002, Mol. Microbiol., 44, 335-349) described the concept of CBDs determining the specific recognition and high-affinity binding to bacterial cell wall carbohydrates using Listeria monocytogenes as a role model. US 2004/0197833 teaches the use of immobilized isolated CBDs in a method for the enrichment of target cells.

The object of the present invention is to provide improved and advantageous proteins which allow the reliable detection and enrichment and/or lysis of bacterial cells.

The object is solved by the subject matter as defined in the claims.

The following figures illustrate the present invention.

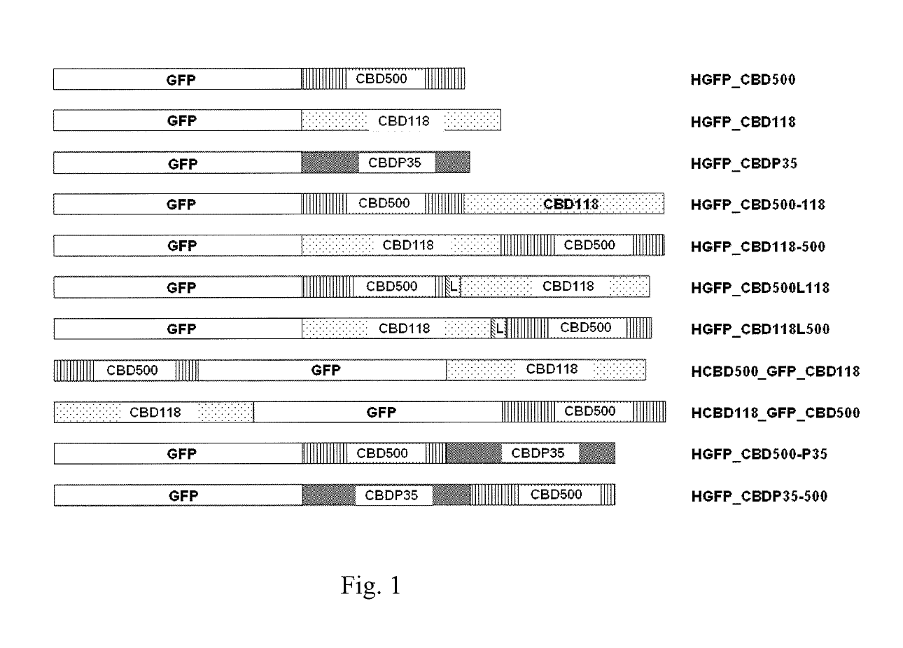

FIG. 1: Schematic representation of the GFP-double CBD fusion proteins against Listeria cells, as well as the GFP-single CBD constructs serving as references. GFP=Green fluorescent protein; CBD500, CBD118, CBDP35 =Cell wall binding domains of Listeria phage endolysins Ply500, Ply118, and PlyP35, respectively; L=linker region of the PlyPSA endolysin.

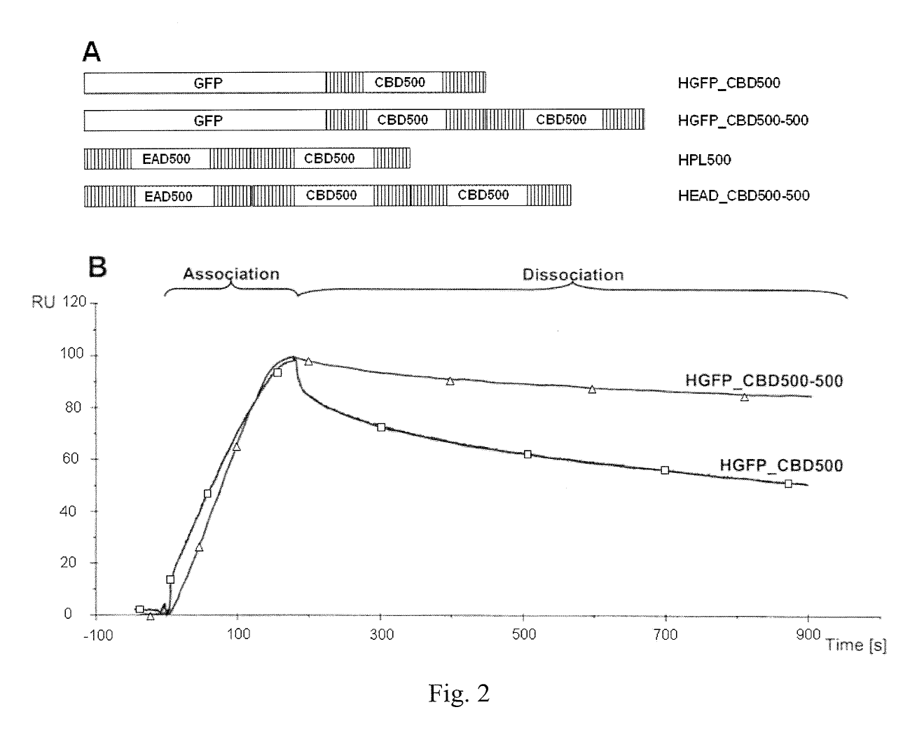

FIG. 2: Peptidoglycan binding proteins with duplicated CBD resulting in higher affinity due to reduced dissociation from the cell wall. (A) Schematic representations of double CBD500 fusion proteins as well as the respective single CBD500 constructs serving as references. GFP=Green fluorescent protein; EAD500=Enzymatically active domain of Listeria phage endolysin Ply500; CBD500=Cell wall binding domain of Ply500. (B) Overlay of the SPR sensograms of HGFP_CBD500 (black) and HGFP_CBD500-500 (grey), measured at a concentration of 50 nM. Association and dissociation phases are indicated. RU=relative response units.

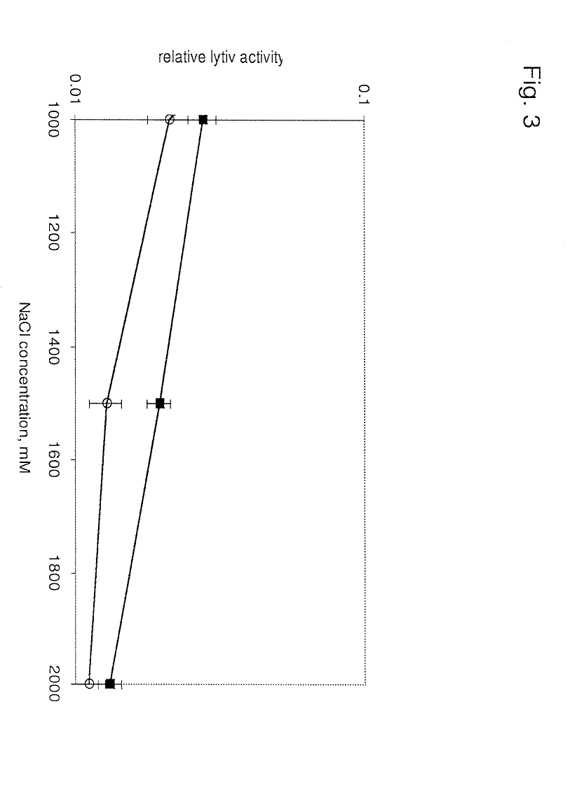

FIG. 3: Relative lytic activities of wild-type ply500 with N-terminal His-tag (open circles) and H_EAD_CBD500-500 (solid squares) against cells of Listeria monocytogenes WSLC 1042 measured with the photometric lysis assay at different NaCl concentrations. The optimum activity of wild-type ply500 at 200 mM NaCl corresponds to 1.0. All assays were carried out in triplicate.

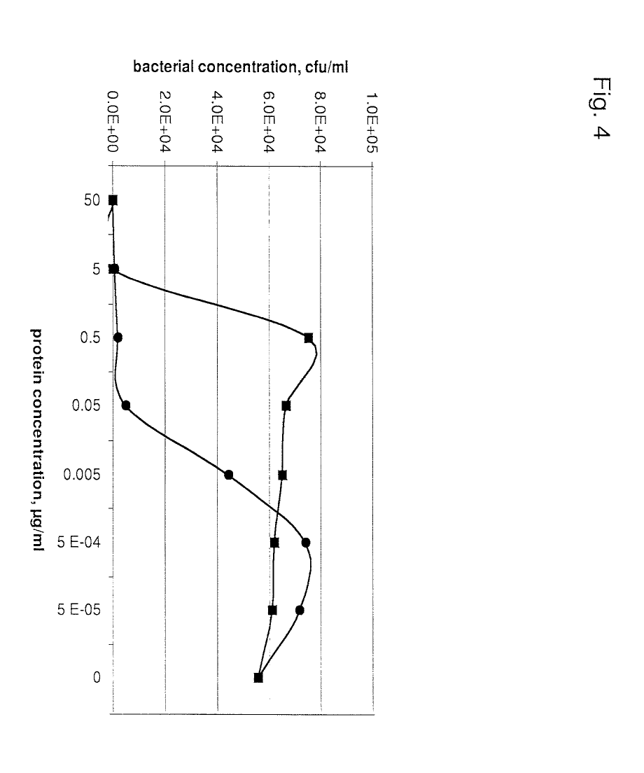

FIG. 4: Determination of the minimal bactericidal concentration (MBC) of peptidoglycan lysing enzymes against enterococci. The bacterial concentration of surviving cells of Enterococcus faecalis strain 17 is shown in dependence of the protein concentration of Fab25 VL (squares) or EADFab25_CBD25_CBD₂₀ (circles) present in the cell lysis assay.

The term “peptidoglycan lysing enzyme” as used herein refers to an enzyme which is suitable to lyse bacterial cell walls. The enzyme comprises at least one of the following activities of which the “enzymatically active domains” (EADs) of the peptidoglycan lysing enzymes are constituted: endopeptidase, N-acetyl-muramoyl-L-alanine-amidase (amidase), N-acetyl-muramidase (lysozyme or lytic transglycosylase) or N-acetyl-glucosaminidase. Either, the enzyme is phage or prophage encoded, the so-called “endolysins” or it is derived from related cell wall lysing enzymes coded by bacteria, the so-called “autolysins” or other bacterial peptiglycan lysing enzymes like bacteriocins, virulence factors or other antimicrobial polypeptides (e.g. lysostaphin, ALE-1 lysin, mutanolysin, enterolysin). In addition, the peptidoglycan lysing enzymes contain also regions which are enzymatically inactive, and bind to the cell wall of the host bacteria, the so-called CBDs (cell wall binding domains).

The term “peptidoglycan binding protein” as used herein refers to an artificially constructed bacterial cell binding protein which has none of the enzymatic activities described for the peptidoglycan lysing enzyme. The peptidoglycan binding protein comprises more than one CBD derived from a CBD. The peptidoglycan binding protein is constructed by shuffling of naturally occurring CBDs and/or by multiplication of naturally occurring CBDs.

The term “domain” as used herein refers to a subunit of a peptidoglycan lysing enzyme which is ascribed a specific function, and can also coincide with a structural or evolutionary conserved domain. Specific functions associated with a domain are for example bacterial peptidoglycan lysis or bacterial cell binding. The functional domains are sometimes also called “modules”.

The term “CBD” as used herein refers to the cell wall binding domain of a peptidoglycan lysing enzyme, which is often found at the C-terminus of the protein. CBD domains have no enzymatic activity in terms of hydrolyzing the cell wall, but mediate binding of the peptidoglycan lysing enzyme to the bacterial cell wall. The term CBD as used herein describes a segment within a polypeptide chain which is derived from a naturally occurring peptidoglycan lysing enzyme.

The term “EAD” as used herein refers to the enzymatically active domain of a peptidoglycan lysing enzyme which is responsible for hydrolysis of the bacterial peptidoglycan. It contains at least one of the enzymatic activities described for a peptidoglycan lysing enzyme. The term EAD as used herein describes a segment within a polypeptide chain which is derived from a naturally occurring peptidoglycan lysing enzyme.

A “CHAP” domain (cysteine, histidine-dependent amidohydrolases/peptidases) is a region of about 110 to about 140 amino acid residues that is found in proteins from bacteria, bacteriophages, archaea and eukaryotes of the Trypanosomidae family. The proteins may function mainly in peptidoglycan hydrolysis. The CHAP domain is commonly associated with bacterial type SH3 domains and with several families of amidase domains. CHAP domain containing proteins may utilize a catalytic cysteine residue in a nucleophilic-attack mechanism. The CHAP domain contains two invariant amino acid residues, a cysteine and a histidine residue. These residues form part of the putative active site of CHAP domain containing proteins.

The term “ami” as used herein describes an enzymatically defined domain which exhibits amidase activity, i.e. it hydrolyzes the amide bond between N-acetylmuramine in the peptidoglycan backbone and the adjacent amino acid residue which is usually L-ala in the peptide linker. The amidase are often metal ion dependent for activity.

The term “SH3” domain which is sometimes also called Src homology 3 domain as used herein describes a small non-catalytic protein domain of about 60 amino acid residues which is characteristic for proteins which interact with other binding partners. It is identified via a proline-rich consensus motif. The SH3 domain is located within the CBD. SH3 domains found in peptidoglycan lysing enzymes are often of the SH3b or SH3_{—}5 type.

The term “wild-type” refers to the naturally occurring form of a protein or a nucleic acid with respect to the sequence.

The term “shuffling” as used herein refers to the combination of different fragments of polypeptides from different wild-type enzymes into new chimaeric polypeptide constructs. In this context, the enzymes are preferentially peptidoglycan lysing enzymes, and the fragments are preferentially EADs and CBDs. Usually, the fragments are combined by molecular biological methods on nucleic acid level. Additional linker sequences may be introduced between the fragments for structural or cloning reasons.

One object of the present invention refers to peptidoglycan lysing enzymes that are composed of at least one EAD and at least two CBDs. Artificially created peptidoglycan lysing enzymes according to the invention exhibit new properties like an extended or altered binding range compared to naturally occurring proteins or an increased binding affinity to the bacterial cell wall or an increased or altered lytic activity or combinations thereof.

Another object of the present invention refers to peptidoglycan binding proteins that are composed of at least two CBDs. Artificially created peptidoglycan binding proteins according to the invention exhibit new properties like an extended or altered binding range compared to naturally occurring proteins or an increased binding affinity to the bacterial cell wall or both.

In the peptidoglycan lysing enzymes or peptidoglycan binding proteins according to the invention the at least two CBDs may be derived from two different peptidoglycan lysing enzymes (domain shuffling) or by multiplication of one CBD naturally occurring in an endolysin. If more than one EAD is present in the peptidoglycan lysing enzyme according to the invention the EADs may be derived from two different peptidoglycan lysing enzymes.

Meanwhile, a large number of peptidoglycan lysing proteins against different genera, species or strains of gram positive and gram negative bacteria is described in the art. The modular nature of the peptidoglycan lysing proteins, and the distinction between EAD and CBD is well known. Lots of conserved domains existing in peptidoglycan lysing proteins are characterized functionally, and their existence within a polypeptide or nucleotide sequence can be predicted by suitable computer programs which use respective protein or nucleic acid databases, e.g. CDD (Marchler-Bauer et al., 2005; Nucleic Acids Research, 33, D192-D196); Pfam (Finn et al., 2006, Nucleic Acids Research 34, D247-D251) or SMART (Schultz et al., 1998, Proc. Natl. Acad. Sci. USA 95, 5857-5864, Letunic et al., 2006, Nucleic Acids Res 34, D257-D260) or by binding assays with deletion mutants (Loessner et al., 2002, Mol. Microbiol., 44, 335-349). The artificial peptidoglycan lysing enzymes according to the invention are constructed by combining the desired enzymatic activity derived from an EAD with at least two CBDs for the cell binding activity using standard techniques for cloning and production of recombinant proteins as described in Sambrook et al. (Molecular cloning. A laboratory manual; 2nd ed. Cold Spring Harbor Laboratory Press 1989). The artificial peptidoglycan binding proteins according to the invention are constructed by combining at least two CBDs for the cell binding activity using standard techniques for cloning and production of recombinant proteins as described in Sambrook et al. (Molecular cloning. A laboratory manual; 2nd ed. Cold Spring Harbor Laboratory Press 1989). The at least two CBDs can derive from different peptidoglycan lysing enzymes which leads to shuffled chimaeric enzymes, or they can derive from a multiplication of CBDs from one naturally occurring enzyme, or combinations of both. Principally, all naturally occurring peptidoglycan lysing enzymes are potential candidates for the supply of EAD and CBD domains.

The peptidoglycan lysing enzymes preferably comprise at least one EAD selected from the group composed of Amidase_{—}5 (bacteriophage peptidoglycan hydrolase, pfam05382), Amidase_{—}2 (N-acetylmuramoyl-L-alanine amidase, pfam01510), Amidase_{—}3 (N-acetylmuramoyl-L-alanine amidase, pfam01520), Transgly (transglycosylase, pfam00912), Peptidase_M23 (peptidase family M23, pfam01551), endolysin_autolysin (CD00737), Hydrolase_{—}2 (cell wall hydrolase, pfam07486), CHAP (amidase, pfam05257), Transglycosylase (transglycosylase like domain, pfam06737), MtlB (membrane-bound lytic murein transglycosylase B, COG2951), MtlA (membrane-bound lytic murein transglycosylase A, COG2821), MtlE (membrane-bound lytic murein transglycosylase E, COG0741), bacteriophage_lambdalysozyme (lysis of the bond between N-acetylmuramic acid and N-acetylglucosamine, CD00736), Peptidase_M74 (penicillin-insensitive murein endopeptidase, pfam03411), SLT (transglycosylase SLT, pfam01464), Lys (C-type lysozyme/alpha-lactalbumin family, pfam00062), COG5632 (N-acetylmuramoyl-L-alanine amidase, COG5632), MepA (murein endopeptidase, COG3770), COG1215 (glycosyltransferase, COG1215), AmiC(N-acetylmuramoyl-L-alanine amidase, COG0860), Spr (cell wall-associated hydrolase, COG0791), bacteriophage_T4-like_lysozyme (lysis of the bond between N-acetylmuramic acid and N-acetylglucosamine, cd00735), LT_GEWL (lytic transglycosylase (LT) and goose egg white lysozyme (GEWL) domain, cd00254), peptidase_S66 (LD-carboxypeptidase, pfam02016), Glyco_hydro_{—}70 (glycosyl hydrolase family 70, pfam02324), Glyco_hydro_{—}25 (glycosyl hydrolase familiy 25), VanY (D-alanyl-D-alanine carboxypeptidase, pfam02557), and LYZ2 (lysozyme subfamily 2, smart 00047).

The peptidoglycan lysing enzymes preferably comprise at least one CBD selected from the group composed of SH3_{—}5 (bacterial SH3 domain, pfam08460), SH3_{—}4 (bacterial SH3 domain, pfam06347), SH3_{—}3 (bacterial SH3 domain, pfam08239), SH3b (bacterial SH3 domain homologue, smart00287), LysM (LysM domain found in a variety of enzymes involved in cell wall degradation, pfam01476 and cd00118), PG_binding_{—}1 (putative peptidoglycan binding domain, pfam01471), PG_binding_{—}2 (putative peptidoglycan binding domain, pfam08823), MtlA (peptidoglycan binding domain from murein degrading transglycosylase, pfam03462), Cpl-7 (C-terminal domain of Cpl-7 lysozyme, pfam08230), CW_binding_{—}1 (putative cell wall binding repeat, pfam01473), LytB (putative cell wall-binding domain, COG2247), and LytE (LysM repeat, COG1388).

Preferably, the domains described above have amino acid residue lengths in the range of about 15 to about 250 amino acid residues, preferred are lengths in the range of about 20 to about 200 amino acid residues. As an example, about 15 to about 40 amino acid residue long domains are found in peptidoglycan binding domains like the LysM domain or the CW_binding_{—}1 motif which is responsible for cholin binding. These small domains are often found as naturally repeated motifs also in wild-type cell wall lysing enzymes. These domains can be combined with additional CBDs from other cell wall lysing enzymes in order to create chimaeric shuffled artificial peptidoglycan lysing enzymes or peptidoglycan binding proteins.

Usually, complete EAD or CBD domains of peptidoglycan lysing enzymes are larger than the conserved domains described above. Preferentially, an EAD or CBD is in the range of about 50 residues to about 400 residues long. Each EAD and CBD contains at least one functional domain in order to exhibit their functions of peptidoglycan lysis or bacterial cell binding, but can also comprise more than one functional domain and additional sequence segments with unknown function. EAD and CBD domains of peptidoglycan binding enzymes are not always defined by the conserved domains described above. There are also peptidoglycan binding enzymes known (e.g. Ply118) which bind and lyse bacterial cells although none of the above described conserved domains is found. Whether potential domains function as an EAD or CBD can be tested with suitable functional assays (e.g. photometric lysis assay, plate lysis assay or determination of minimal bactericidal concentration (MBC) for peptidoglycan lysis (EAD), and cell binding assay, fluorescence microscopy or determination of binding affinity for cell binding (CBD). The domain borders of EADs and CBDs can be defined by local alignment search tools (e.g. BLAST at the NCBI, Altschul et al., 1997, Nucleic Acids Res. 17, 3389-3402) which find regions of local similarity between sequences. In addition, a multitude of peptidoglycan lysing enzymes are already described with respect to their EAD and CBD domains.

Preferably, the peptidoglycan lysing enzymes and peptidoglycan binding proteins of the present invention are composed of EADs and CBDs derived from wild-type peptidoglycan lysing enzymes selected from the group consisting of Ply500, Ply511, Ply118, Ply100, PlyP40, Ply3626, phiLM4 endolysin, PlyCD119, PlyPSAa, Ply21, PlyBA, Ply12, PlyP35, PlyPH, PlyL, PlyB, phi11 endolysin, phi MR11 endolysin, phi12 endolysin, S. aureus phage PVL amidase, plypitti26, ΦSA2usa endolysin, endolysin of Staphylococcus warneri M phage ΦWMY PlyGBS, B30 endolysin, Cpl-1, Cpl-7, Cpl-9, PlyG, PlyC, pal amidase, Fab25, Fab20, endolysins from the Enterococcus faecalis V583 prophage, lysostaphin, phage PL-1 amidase, S. capitis ALE-1 endopeptidase, mutanolysin (N-acetylmuramidase of Streptomyces globisporus ATCC 21553), enterolysin A (cell wall degrading bacteriocin from Enterococcus faecalis LMG 2333), LysK, LytM, Ami autolysin from L. monocytogenes, endolysins of the Pseudomonas aeruginosa phages ΦKZ and EL, T4 lysozyme, gp61 muramidase, and STM0016 muramidase.

The wild-type peptidoglycan lysing enzyme PlyP40 has a length of 344 amino acid residues in its wild type form. It possesses two functional domains that have only a minimal homology with other known endolysins. The N-terminal amino acid residues at the positions from 1 to 200 represent the enzymatically active domain (EAD) which is depicted in SEQ ID NO: 103. The cell binding domain (CBD) of PlyP40 comprises the C-terminal located amino acid residues from 227 to 344 which are depicted in SEQ ID NO: 104. Thus, the EAD deriving from the wild-type peptidoglycan lysing enzyme PlyP40 comprises preferably an amino acid sequence according to SEQ ID NO: 103, whereas the CBD deriving from the wild-type peptidoglycan lysing enzyme PlyP40 comprises preferably an amino acid sequence according to SEQ ID NO: 104.

The fragments derived from naturally occurring peptidoglycan lysing enzymes in order to construct the enzymes and proteins according to the invention may not combine the mere sequence segments determined from the prediction of the conserved functional domains as described above, but preferably add suitable linker sequences which connect the different functional modules. The linker sequences can be derived from the wild-type sequences in neighborhood to the defined functional domains or can be external suitable linker sequences known from the art. A suitable linker is for example the short domain linker with the sequence AAKNPN or TGKTVAAKNPNRHS (SEQ IDs No: 61 and 11) from the Listeria endolysin PlyPSA (Korndörfer et al., 2006, J. Mol. Biol., 364, 678-689) defined from the x-ray structure. Polyglycine linkers are also known in the art to serve as flexible domain linkers. Preferred linkers are also glycine and alanine rich linkers. Specific sequences for glycine and alanine rich linkers are given as SEQ ID NO:63, 64 and 65. Preferred are also proline and threonine rich sequences which occur as natural linkers, e.g. in enterolysin A SEQ ID NO:66. Proline and threonine rich linker sequences can be described by the consensus motif (PT)_{X}P or (PT)_{X}T, where x stands for an integer in the range of 1 to 10. Another linker possibility are the so-called “junction zones” between EADs and CBDs described in Croux et al. (1993, Molec. Microbiol., 9, 1019-1025. A skilled person knows several methods how to predict a suitable boundary for a functional domain to be taken out of a wild-type enzyme, e.g. secondary structure prediction, prediction of domain linkers, inspection of 3D-models of proteins or inspection of domain linkers and boundaries in highly resolved X-ray and NMR structures of proteins. Suitable methods are for example described in Garnier et al., 1996, Methods in Enzymology 266, 540-553; Miyazaki et al., 2002, J. Struct. Funct. Genomics, 15, 37-51; George and Hering a, 2003, Protein Eng. 15, 871-879; Bae et al., 2005, Bioinformatics, 21, 2264-2270, Altschul et al., 1997, Nucleic Acids Res. 17, 3389-3402; Schwede et al., 2003, Nucleic Acids Research 31, 3381-3385. Lund et al, CPHmodels 2.0: X3M a Computer Program to Extract 3D Models. Abstract at the CASP5 conferenceA102, 2002. The length of polypeptide linker between EAD and CBD domains or between CBD and CBD domains are in the range of about 5 to about 150 amino acid residues, preferentially of about 6 to about 60 amino acid residues.

Preferably, the order for the combination of EAD and CBDs in the peptidoglycan lysing enzymes according to the invention is EAD-CBD1-CBD2(-CBDN, N=3 or more) from the N-terminus to the C-terminus. Preferred are also variants where an at least second EAD is added next to the EAD at the N-terminus or at the C-terminus. Preferred are also variants where the at least two CBDs are positioned at the N-terminus or at the N- and C-terminus with the EADs positioned in the middle. In addition, marker sequences or tags can be included, which can both be positioned N-terminal, C-terminal or in the middle, but especially preferred at the N-terminus.

Peptidoglycan lysing enzymes and peptidoglycan binding proteins according to the invention exhibit new properties compared to the wild-type enzymes from which they are derived.

The binding range of a peptidoglycan lysing enzyme or peptidoglycan binding protein determines the bacterial host range which is recognized. Most of the naturally occurring peptidoglycan lysing enzymes exhibit a relatively narrow host range. For technical application of the peptidoglycan lysing enzymes or peptidoglycan binding proteins it is often advantageous to extend the host range of the proteins so that an increased number of bacterial strains or species can be killed, captured or detected depending on the respective application. An extended host range comprised within one protein avoids the use of two or more proteins for the same application which has the advantages of reduced costs for protein production, reduced effort to optimize conditions for different proteins, simpler medical approval proceedings, and reduced immunogenicity. An extended host range which combines for example Staphylococci and Enterococci is useful in the therapy or prevention of nosocomial infections where multiresistant strains of both genera are an increasing problem. An extended host range is also useful in bacterial detection or a method to remove harmful bacteria from food. For example, pathogenic strains are found within all serovars of Listeria. None of the naturally occurring Listeria endolysins, however, is able to lyse cells from all serovars. A peptidoglycan lysing enzyme combining more than one CBD according to the invention is able to lyse all serovars. A host range which is not extended, but somehow altered compared to naturally occurring proteins, may be useful for applications which need tailored proteins for a given set of bacterial cells to be lysed, captured or detected. The binding range of peptidoglycan lysing enzymes or peptidoglycan binding proteins can be determined with assays known from the art or with the plate lysis assay, photometric lysis assay, binding assay or fluorescence microscopy described in the examples.

An increased binding affinity of peptidoglycan lysing enzymes or peptidoglycan binding proteins compared to wild-type proteins helps to reduce the amount of protein needed for any technical application which relies on the binding of the bacterial cells like cell lysis, cell capture, and detection. This reduces costs and minimizes immunological reactions and potential side effects in therapeutical applications. In applications relying on bacterial cell capture, the assays are less sensitive for washing steps which decreases background signals, incubation times can be reduced, and detection assays are more sensitive. An increased binding affinity can be measured with assays known from the art or with the surface plasmon resonance analysis or the assay for determination of the minimal bactericidal concentration described in the examples.

An increased lytic activity of peptidoglycan lysing enzymes compared to wild-type enzymes is useful in all applications relying on the lysis of bacterial cells like protection and therapy of infections, sanitation, cell lysis as an initial step in bacterial detection, or removal of pathogenic bacteria from food, feed, cosmetics etc. The amount of protein needed for the respective application is reduced compared to the wild-type protein which reduces costs and minimizes immunological reactions and potential side effects in therapeutical applications. An altered lytic activity compared to wild-type could be for example a different pH-optimum of the artificial enzyme or a higher lysis activity at other buffer compositions (e.g. high ionic strength, activity in the presence of organic solvents, activity in the presence of specific ions). This also includes a higher activity in specific samples like blood, human serum, or other medical samples. A pH-optimum of an artificial enzyme which is shifted to lower pH is for example interesting for an application of the artificial enzyme in food industry as food products or intermediate products in food processing often have a low pH-value, e.g. in dairy farming. Enzyme function under high salt concentration is also important in food industry, e.g. in cheese production. An increased or altered lytic activity can be determined with assays known from the art or with the plate lysis assay, photometric lysis assay, or the assay for determination of the minimal bactericidal concentration described in the examples.

In one aspect the present invention relates to artificial peptidoglycan lysing enzymes and peptidoglycan binding proteins which can be used to lyse, capture and/or detect Listeria bacteria. The inventors combined domains of the Listeria endolysins ply500 (SEQ ID NO:1), ply118 (SEQ ID NO:3) and plyP35 (SEQ ID NO:5) using the method described above in order to create artificial peptidoglycan lysing enzymes and peptidoglycan binding proteins which exhibit new properties compared to the wild-type enzymes. Ply500 comprises a conserved D-alanyl-D-alanine carboxypeptidase (VanY; pfam02557) domain as an EAD. The CBD of ply500 begins with an amino acid residue in the range of about H133 to Q150 and ends with K289. For Ply118 no conserved domains were found within the amino acid sequence. From sequence alignments with homologous peptidoglycan lysing enzymes, however, it was derived that the CBD of ply118 begins with an amino acid residue in the range of about D90 to K180 and ends with amino acid residue K289. Preferred N-terminal starting amino acid residues for CBD118 are D90, K100, G127, 5151, N161 or K180. PlyP35 also comprises a conserved D-alanyl-D-alanine carboxypeptidase (VanY; pfam02557) domain as an EAD. The CBD of plyP35 begins with an amino acid residue in the range of about P130 to N156 and ends with an amino acid residue in the range of Y281 to K291. Preferred N-terminal starting amino acid residues for CBDP35 are P130, A134, K143 and N156. Preferred C-terminal amino acid residues for CBDP35 are Y281, L286, and K291.

Preferred peptidoglycan binding proteins according to the invention are CBD500-118 (SEQ ID NO:7) which comprise the CBD of ply500 (amino acid residues H133 to K289) in the N-terminal position and the CBD of ply118 (amino acid residues D90 to I281) in the C-terminal position connected without an additional linker sequence, and CBD500L118 (SEQ ID NO:9) which comprises the CBD of ply500 (amino acid residues Q150 to K289) and the CBD of ply118 (amino acid residues K100 to I281) with a linker (L) connecting the two domains. The domain linker in this case is the plyPSA linker region with the amino acid sequence TGKTVAAKNPNRHS (SEQ ID NO:11), which correspond to amino acid residues 173 to 186 from the Listeria endolysin PlyPSA (Korndörfer et al., 2006, J. Mol. Biol., 364, 678-689).

Further preferred embodiments according to the invention are the artificial peptidoglycan binding proteins CBD118-500 (SEQ ID NO:13) which comprise the CBD of ply500 (amino acid residues H133 to K289) in N-terminal position and the CBD of ply118 (amino acid residues D90 to I281) in C-terminal position connected without an additional linker sequence, and CBD118L500 (SEQ ID NO:15) which comprises the CBD of ply118 (amino acid residues K100 to I281) and the CBD of ply500 (amino acid residues Q150 to K289) with a linker (L) connecting the two domains. The domain linker in this case is the plyPSA linker region (SEQ ID NO:11).

The peptidoglycan binding proteins CBD500-118, CBD500L118, CBD118-500, and CBD118L500 all exhibit altered cell binding activities with respect to host range and binding activity compared to the wild-type enzymes ply500 and ply118 from which the CBD domains were derived. The cell binding activity of the constructs CBD500L118 and CBD118L500 discloses that a linker between two domains helps to achieve an extended host range which combines the binding specificities of the wt-enzymes.

Further preferred peptidoglycan binding proteins according to the present invention are CBD500-P35 (SEQ ID NO:17) which comprises the CBD of ply500 (amino acid residues Q150 to K289) in N-terminal position and the CBD of plyP35 (amino acid residues P130 to K291) in C-terminal position, and the protein with the inverse orientation of CBDs CBDP35-500 (SEQ ID NO:19) which comprises the CBD of plyP35 (amino acid residues P130 to K291) at the N-terminus, and the CBD of ply500 (amino acid residues Q150 to K289) at the C-terminus. In this case, the CBDs are not connected by an external linker sequence, as the fragment for the CBD of plyP35 includes the internal domain linker of plyP35.

The artificial peptidoglycan binding proteins CBD500-P35 and CBDP35-500 both exhibit an extended host range compared to the wild-type enzymes ply500 and plyP35 from which the CBD domains were derived. Both chimaeric proteins combine the different binding specificities of the two wt-enzymes within one protein. The orientation of the CBDs makes no difference in this case. Both CBDs can be positioned N-terminally as well as C-terminally.

Further preferred peptidoglycan binding proteins according to the present invention are CBD500-500 (SEQ ID NO:21) which exhibits a duplication of the CBD of ply500 (amino acid residues Q150 to K289), and the artificial peptidoglycan lysing enzyme EAD-CBD500-500 (SEQ ID NO:23) which exhibits a duplication of the naturally occurring CBD in ply500.

Both proteins according to the invention exhibit a higher binding affinity to Listeria cells compared to the wild-type, and EAD-CBD500-500 in addition exhibits an increased lysis activity under high salt conditions compared to ply500.

In another aspect the present invention relates to artificial peptidoglycan lysing enzymes which can be used to lyse, capture or detect Enterococcus bacteria. The inventors combined domains of the Enterococcus endolysins Fab25VL (SEQ ID NO:25) and Fab20VL (SEQ ID NO:27) using the method described above in order to create artificial peptidoglycan lysing enzymes and peptidoglycan binding proteins which exhibit new properties compared to the wild-type enzymes.

Fab25VL is an endolysin of 317 amino acid residues length which preferentially binds and lysis bacteria from the species E. faecium, but also some strains from the species E. faecalis. The N-terminally positioned EAD of Fab25VL (amino acid residues 1 to 167) exhibits a conserved Amidase_{—}2 domain which functions as an N-acetylmuramoyl-L-alanine-amidase. The CBD comprises the amino acid residues 200 to 317. Between the two domains, a linker region comprising amino acid residues 168 to 199 is observed. Fab20VL is an endolysin of 365 amino acid residues length which preferentially binds and lysis bacteria from the species E. faecalis. The N-terminally positioned EAD (amino acid residues 40 to 194) of Fab20VL also exhibits a conserved Amidase_{—}2 domain which functions as an N-acetylmuramoyl-L-alanine-amidase. The CBD of Fab20VL (amino acid residues 215 to 365) comprises a bacterial SH3 domain in its C-terminal part which shows homologies to peptidoglycan lysing enzymes from Staphylococcus and Streptococcus phages. An N-terminally truncated variant of Fab20VL with a deletion of amino acid residues 1 to 19—Fab20K (SEQ ID NO:29) was constructed which showed better expression in E. coli compared to Fab20VL.

A preferred peptidoglycan lysing enzyme according to the present invention is SEQ ID NO:31 which combines the EAD and CBD of Fab25 (amino acid residues 1 to 317) with the CBD of Fab20VL (amino acid residues 215 to 365) and a short linker segment derived from Fab20VL (amino acid residues 200 to 214). The construct is denoted EADFab25_CBD25_CBD20. EADFab25_CBD25_CBD₂₀ exhibits an extended host range, an increased lysis activity with respect to living cells and an increased binding affinity as new features compared to the wt-enzymes.

Further preferred peptidoglycan binding enzymes according to the present invention are composed of at least two EADs and at least two CBDs capable of detecting and binding Staphylococcus bacteria.

Preferably the peptidoglycan lysing proteins according to the present invention comprise tags such as His-tag (Nieba et al., 1997, Anal. Biochem., 252, 217-228), Strep-tag (Voss & Skerra, 1997, Protein Eng., 10, 975-982), Avi-tag (U.S. Pat. No. 5,723,584; U.S. Pat. No. 5,874,239), Myc-tag (Evan et al., Mol & Cell Biol, 5, 3610-3616), GST-tag (Peng et al. 1993, Protein Expr. Purif., 412, 95-100), JS-tag (WO 2008/077397), cystein-tag (EP1399551, SEQ IDs No:6 and 7), HA-tag (amino acid sequence EQKLISEEDL), FLAG-tag (Hopp et al., Bio/Technology. 1988; 6:1204-1210) or other tags known in the art. Preferably the tag is coupled to the C-terminus or the N-terminus of the peptidoglycan lysing protein according to the invention, most preferably to the N-terminus. Tags can be useful to facilitate expression and/or purification of the peptidoglycan lysing protein, to immobilize the peptidoglycan lysing protein according to the invention to a surface or to serve as a marker for detection of the peptidoglycan lysing protein, e.g. by antibody binding in different ELISA assay formats.

Preferably the peptidoglycan lysing proteins according to the invention comprise marker or label moieties such as biotin, streptavidin, GFP (green fluorescent protein), YFP (yellow fluorescent protein), cyan fluorescent protein, RedStar protein or other fluorescent markers, alkaline phosphatase, horse radish peroxidase, immuno-gold labels, spin labels or other markers and labels known in the art. The markers can be attached in a recombinant way if they are of polypeptide nature or post-translationally by chemical modification of the polypeptide residues. The markers or labels are especially useful to detect the peptidoglycan lysing proteins according to the invention when they are used in diagnostics.

Further preferred peptidoglycan lysing proteins are the constructs HGFP-CBD118-500 (SEQ ID NO:33), HGFP-CBD500-118 (SEQ ID NO:35), HGFP-CBD118L500 (SEQ ID NO:37), HGFP-CBD500L118 (SEQ ID NO:39), HGFP-CBD500-P35 (SEQ ID NO:41), HGFP-CBDP35-500 (SEQ ID NO:43), HCBD500-GFP-CBD118 (SEQ ID NO:45), HCBD118-GFP-CBD500 (SEQ ID NO:47), HGFP-CBD500-500 (SEQ ID NO:49), and HEAD-CBD500-500 (SEQ ID NO:51). H denotes a his-tag including six histidines (SEQ ID NO:53), and GFP denotes green fluorescent protein introduced as a fluorescent marker (SEQ ID NO:55).

All of the above mentioned fusion constructs including a his-tag and a GFP marker show Listeria cell binding activity and the peptidoglycan lysing enzymes additionally lysis activity. The constructs HCBD500-GFP-CBD118 and HCBD118-GFP-CBD500 however show that an N-terminal position of the GFP marker is preferred compared to a positioning between the two CBD domains, as the cell binding activity, especially the cell binding activity of CBD118, is reduced in these constructs.

In summary, the object of the present invention is to alter and improve the properties of wild-type peptidoglycan lysing enzymes by artificial combination of functional domains by shuffling or by multiplication of naturally occurring domains. Peptidoglycan lysing enzymes according to the invention are composed of at least one EAD and at least two CBDs in order to extend the binding range of naturally occurring proteins, and/or to increase the binding affinity to the bacterial cell wall, and/or to increase or modify the lytic activity. Peptidoglycan binding proteins according to the invention are composed of at least two CBDs in order to extend the binding range of naturally occurring proteins, and/or to increase the binding affinity to the bacterial cell wall. In the polypeptides according to the invention the at least two CBDs are derived from two different peptidoglycan lysing enzymes (domain shuffling) or by multiplication of naturally occurring CBDs.

Preferred is a recombinant polypeptide as depicted in SEQ ID NO: 7, 9, 13, 15, 17, 19, 21, 23, 31, 33, 35, 37, 39, 41, 43, 45, 47, 49, 51, 71, 73, 75, 77, 79, 81, 83, 85, 87, 89, 91, 93, 95, 97, 99, 101.

In another preferred embodiment of the present invention the recombinant polypeptides according to the present invention as listed above comprise modifications and/or alterations of the amino acid sequences. Such alterations and/or modifications may comprise mutations such as deletions, insertions and additions, substitutions or combinations thereof and/or chemical changes of the amino acid residues, e.g. biotinylation, acetylation, pegylation, chemical changes of the amino-, SH- or carboxyl-groups. Said modified and/or altered recombinant polypeptides exhibit the activity of the single domains of the respective recombinant polypeptide as listed above. However, said activity of the single domains can each be higher or lower as the activity of the single domains of the respective recombinant polypeptide as listed above. In particular said activity of the single domains can be about 10, 20, 30, 40, 50, 60, 70, 80, 90, 100, 110, 120, 130, 140, 150, 160, 170, 180, 190, 200% or more of the activity of the single domains of the respective recombinant polypeptide as listed above. The activity of the single domains can be measured by the assays as already described herein for measuring the activity of the CBDs and EADs.

In a further aspect the present invention relates to a nucleic acid molecule comprising a nucleotide sequence encoding the polypeptides according to the present invention.

Preferred is a nucleic acid molecule, wherein the nucleic acid molecule comprises a nucleotide sequence as depicted in SEQ ID NO: 8, 10, 14, 16, 18, 20, 22, 24, 32, 34, 36, 38, 40, 42, 44, 46, 48, 50, 52, 72, 74, 76, 78, 80, 82, 84, 86, 88, 90, 92, 94, 96, 98, 100, 102.

In a further aspect the present invention relates to a vector comprising a nucleic acid sequence of the invention. Preferably, said vector provides for the expression of said polypeptide of the invention in a suitable host cell. Said host cell may be selected due to mere biotechnological reasons, e.g. yield, solubility, costs, etc. but may be also selected from a medical point of view, e.g. a non-pathological bacteria or yeast, human cells, if said cells are to be administered to a subject. Said vector may provide for the constitutive or inducible expression of said polypeptides according to the present invention.

In a further aspect of the present invention the above mentioned polypeptides and/or cells are employed in a method for the treatment or prophylaxis of bacterial infections in a subject, in particular for the treatment or prophylaxis of infections caused by gram positive bacteria like staphylococci (e.g. S. aureus, S. aureus (MRSA), S. epidermidis, S. haemolyticus, S. simulans, S. saprophyticus, S. chromogenes, S. hyicus, S. warneri and/or S. xylosus), enterococci (e.g. Enterococcus faecium, E. faecium (VRE) Enterococcus faecalis), streptococci (Streptococcus pyogenes, S. pneumoniae, S. mutans, S. uberis, S. agalactiae, S. dysgalactiae, Streptococci of the Lancefield groups A, B, C), clostridia (e.g. C. perfringens, C. difficile, C. tetani, C. botulinum, C. tyrobutyricum), bacilli (e.g. Bacillus anthracis, B. cereus), Listeria (e.g. L. monocytogenes, L. innocua), Haemophilus influenza, Corynebacterium diphteriae, Propionibacterium acne, mycobacteria (e.g. Mycobacterium tuberculosis, M bovis). Alternatively, the polypeptides and/or cells according to the invention are employed in a method for the treatment or prophylaxis of bacterial infections in a subject, in particular for the treatment or prophylaxis of infections caused by gram negative bacteria of bacterial groups, families, genera or species comprising strains pathogenic for humans or animals like Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumanii.

Said subject may be a human subject or an animal, in particular animals used in livestock farming and/or dairy farming such as cattle. Said method of treatment encompasses the application of said polypeptide of the present invention to the site of infection or site to be prophylactically treated against infection in a sufficient amount.

In particular said method of treatment may be for the treatment or prophylaxis of infections of the skin, of soft tissues, the respiratory system, the lung, the digestive tract, the eye, the ear, the teeth, the nasopharynx, the mouth, the bones, the vagina, of wounds of bacteremia and/or endocarditis.

In a further preferred embodiment a polypeptide according to the present invention is used in a method of treatment (or prophylaxis) of staphylococcal infections in animals, in particular in livestock and dairy cattle. In particular a polypeptide of the present application is suitable for use in methods of treatment (or prophylaxis) of bovine mastitis, in particular of bovine mastitis caused by S. aureus, S. epidermidis, S. simulans, S. chromogenes, S. hyicus, S. warneri and S. xylosus.

Furthermore, a polypeptide of the present invention may be used prophylactically as sanitizing agent, in particular before or after surgery, or for example during hemodialysis. Similarly, premature infants and immunocompromised persons, or those subjects with need for prosthetic devices can be treated with a polypeptide of the present invention, either prophylactically or during acute infection. In the same context, nosocomial infections, especially by antibiotic resistant strains like Staphylococcus aureus (MRSA), Enterococcus faecium (VRE), Pseudomonas aeruginosa (FQRP) or antibiotica resistant Clostridium difficile may be treated prophylactically or during acute phase with a polypeptide of the present invention. In this embodiment, a polypeptide of the present invention may be used as a disinfectant also in combination with other ingredients useful in a disinfecting solution like detergents, tensids, solvents, antibiotics, lanthibiotics, or bacteriocins.

In a particularly preferred embodiment a polypeptide of the present invention is used for medical treatment, if the infection to be treated (or prevented) is caused by multiresistant bacterial strains, in particular by strains resistant against one or more of the following antibiotics: penicillin, streptomycin, tetracycline, methicillin, cephalothin, gentamicin, cefotaxime, cephalosporin, vancomycin, linezolid, ceftazidime, imipenem or daptomycin. Furthermore, a polypeptide of the present invention can be used in methods of treatment by administering them in combination with conventional antibacterial agents, such as antibiotics, lanthibiotics, bacteriocins other endolysins, etc.

The dosage and route of administration used in a method of treatment (or prophylaxis) according to the present invention depends on the specific disease/site of infection to be treated. The route of administration may be for example in particular embodiments oral, topical, nasopharyngeal, parenteral, intravenous, rectal or any other route of administration.

For application of a polypeptide of the present invention to a site of infection (or site endangered to be infected) a polypeptide of the present invention may be formulated in such manner that the peptidoglycan lysing enzyme is protected from environmental influences such as proteases, oxidation, immune response etc., until it reaches the site of infection.

Therefore, a polypeptide of the present invention may be formulated as capsule, dragee, pill, suppository, injectable solution or any other medical reasonable galenic formulation. In some embodiments these galenic formulation may comprise suitable carriers, stabilizers, flavourings, buffers or other suitable reagents.

For example, for topical application a polypeptide of the present invention may be administered by way of a lotion or plaster.

For nasopharyngeal application a polypeptide according to the present invention may be formulated in saline in order to be applied via a spray to the nose.

For treatment of the intestine, for example in bovine mastitis, suppository formulation can be envisioned. Alternatively, oral administration may be considered. In this case, the polypeptide of the present invention has to be protected from the harsh digestive environment until the site of infection is reached. This can be accomplished for example by using bacteria as carrier, which survive the initial steps of digestion in the stomach and which secret later on a polypeptide of the present invention into the intestinal environment.

All medical applications rely on the effect of the polypeptides of the present invention to lyse specifically and immediately pathogenic bacteria when encountered. This has an immediate impact on the health status of the treated subject by providing a reduction in pathogenic bacteria and bacterial load and simultaneously relieves the immune system. Thus, the major task a person skilled in the art faces is to formulate the polypeptides of the present invention accurately for the respective disease to be treated. For this purpose usually the same galenic formulation as employed for conventional medicaments for these applications can be used.

In a further aspect of the present invention the above mentioned polypeptides and/or cells are a component of a pharmaceutical composition, which optionally comprises a carrier substance.

In an even further aspect the polypeptides and/or cells are part of a cosmetics composition. As mentioned above, several bacterial species can cause irritations on environmentally exposed surfaces of the patient's body such as the skin. In order to prevent such irritations or in order to eliminate minor manifestations of said bacterial pathogens, special cosmetic preparations may be employed, which comprise sufficient amounts of polypeptides of the present invention in order to lyse already existing or freshly settling pathogenic bacteria.

In a further aspect the present invention relates to the use of said polypeptides according to the present invention in foodstuff, on food processing equipment, in food processing plants, on surfaces coming into contact with foodstuff such as shelves and food deposit areas and in all other situations, where pathogenic, facultative pathogenic or other undesirable bacteria can potentially infest food material.

A further aspect the present invention relates to the use of said polypeptides according to the present invention in diagnostics of bacterial infections. In this aspect the polypeptides according to the invention are used as a tool to specifically lyse pathogenic bacteria. The lysis of the bacterial cells by the polypeptides according to the present invention can be supported by the addition of detergents like Triton X-100 or other additives which weaken the bacterial cell envelope like polymyxin B. Specific cell lysis is needed as an initial step for subsequent specific detection of bacteria using nucleic acid based methods like PCR, nucleic acid hybridization or NASBA (Nucleic Acid Sequence Based Amplification), immunological methods like IMS, immunfluorescence or ELISA techniques, or other methods relying on the cellular content of the bacterial cells like enzymatic assays using proteins specific for distinct bacterial groups or species (e.g. β-galactosidase for enterobacteria, coagulase for coagulase positive strains).

Another aspect of the present invention is the use of a peptidoglycan binding protein according to the present invention for binding, enrichment, removing, capture and detection of pathogenic of otherwise undesirable bacteria from a sample. A sample with regard to the methods according to the present invention is any material supposed to or containing bacteria, whereas the bacteria are a target for detection, binding, enrichment, removing or capture. Samples can be e.g. food or feed materials, surface materials or human or veterinary diagnostic probes. Bacteria detection is performed via detection of markers attached to the peptidoglycan binding protein according to the present invention or by detection of said protein itself, e.g. by immunological methods like ELISA. For the methods according to the present invention the peptidoglycan binding proteins according to the present invention may be immobilised on suitable supporting structures, e.g., microtiter plates, test stripes, slides, wafers, filter materials, reaction tubes, magnetic, glass or latex particles, pipette tips or flow-through cell chambers. The supporting structures may consist of, e.g., polystyrene, polypropylene, polycarbonate, PMMA, cellulose acetate, nitrocellulose, glass, silicium wafer, latex. The immobilisation may be accomplished by adsorption, by covalent binding or by further proteins, wherein the covalent binding is preferred. It is relevant that immobilisation is a functional one, that is, said peptidoglycan binding proteins exhibit structures accessible for bacteria although they are bound to the support material.

### EXAMPLES

### Example 1

### DNA Techniques and Cloning Procedures

DNA techniques and cloning procedures according to Sambrook et al. (Molecular cloning. A laboratory manual; 2nd ed. Cold Spring Harbor Laboratory Press 1989) were employed for construction of plasmids coding for endolysin based fusion proteins. The plasmid pQE-30 (QIAGEN) and its derivatives pHGFP, pHGFP_CBD118, pHGFP_CBD500 (Loessner et al. 2002), and pHEADPSA (Korndoerfer et al. 2006), were used as vector backbones for the construction of plasmids coding for N-terminally 6×His-tagged artificial fusion proteins (H stands for His-tag). Restriction sites needed for insertion of the fragments into the plasmids were introduced via the primers. Double CBD fusion constructs were created either by separate amplification of the two CBD fragments and subsequent ligation or alternatively by fusing the two fragments via the PCR based Gene Splicing by Overlap Extension (SOE PCR) method (Horton et al. 1990). CBD118 (SEQ ID NO:57) and CBD500 (SEQ ID NO:58) coding fragments were ligated via EcoRI/MunI sites in both orientations and then inserted into SacI/SalI sites of pHGFP, yielding pHGFP_CBD118-500 and pHGFP_CBD500-118. The plasmids pHGFP_CBDP35-500 and pHGFP_CBD500-P35 were created the same way. In case of pHGFP_CBD118L500 and pHGFP_CBD500L118, the fragment coding for the PlyPSA linker was introduced between the two CBDs by SOE PCR before insertion into pHGFP. As for pHCBD500_GFP_{—}118 and pHCBD118_GFP_{—}500, the 5′ CBD and the GFP fragments were first fused via KpnI sites or by SOE PCR, and then ligated into BamHI/ISacI sites of pHGFP_CBD118 and pHGFP_CBD500, respectively, replacing the mere GFP fragments of these plasmids. For construction of pHGFP_CBD500-500, the CBD500 fragment was cloned into the Sad site of pHGFP_CBD500, resulting in a duplication of CBD500. pHEAD_CBD500-500 was created by inserting the complete ply500 gene into BamHI/SacI sites of pHGFP_CBD500, replacing the GFP fragment. For all constructs, all stop codons except the ones at the 3′ ends were omitted to allow genetic fusions. TAA was generally introduced as stop codon at the 3′ ends. All constructs were verified by nucleotide sequencing.

### Example 2

### Overexpression and Purification of His-Tagged Recombinant Proteins

Overexpression of His-tagged (abbreviated in the respective constructs by “H”) fusion proteins was performed in E. coli XL1-Blue MRF' (Stratagene). The respective strains were grown in modified LB medium (15 g/l tryptose, 8 g/l yeast extract, 5 g/l NaCl) containing 100 μg/ml ampicillin and 30 μg/ml tetracycline for plasmid selection at 30° C., with 0.1 to 1 mM IPTG added as inducer once an OD₆₀₀ of 0.5 was reached. After further incubation at 30° C. for 4 h cultures producing proteins that contain a GFP domain were stored overnight at 4° C. before harvesting and resuspension in 5 ml buffer A (500 mM NaCl, 50 mM Na₂HPO₄, 5 mM imidazole, 0.1% Tween 20, pH 8.0) per 250 ml culture. If no GFP was present, cells were pelleted 4 h after induction. The cells were disrupted by two passages through a French Press 20K cell (SLM Aminco) at 100 MPa, and cell debris was removed by centrifugation and filtration (0.2 μM PES membrane, Millipore).

The 6×His-tagged target proteins in the raw extracts were purified by Immobilized Metal Affinity Chromatography (IMAC) with Ni-NTA Superflow resin (QIAGEN) using Micro Biospin columns (BIORAD). Buffer B (500 mM NaCl, 50 mM Na₂HPO₄, 250 mM imidazole, 0.1% Tween 20, pH 8.0) served as elution buffer. The purified proteins were dialyzed against two changes of dialysis Buffer (100 mM NaCl, 50 mM NaH₂PO₄, 0.005% to 0.1% Tween 20, pH 8.0), filtered (0.2 μM PES membrane, Millipore), and stored at −20° C. after addition of 50% (v/v) of glycerol. For each protein, the course of overexpression and purification was analyzed by SDS-PAGE and the protein concentration was determined spectrophotometrically (NanoDrop ND-1000 Spectrophotometer).

### Example 3

### Binding Assays and Fluorescence Microscopy

The binding properties of GFP-CBD fusion proteins were examined by binding using a representative set of Listeria strains (table 1) of all species and serovars. Late log phase cells of each strain in PBST buffer (50 mM NaH₂PO₄, 120 mM NaCl, pH 8.0, 0.01% Tween 20) were incubated with GFP-CBD protein in excess for 5 mM at room temperature. After washing twice with buffer, the cells were prepared for fluorescence microscopy, using an Axioplan microscope and a filter set with excitation BP 450-490 nm, beamsplitter FT 510 nm, and emission LP 520 nm (Carl Zeiss AG). Pictures of labelled cells were obtained by using a Leica DFC320 camera. For each assay, binding intensity was evaluated by visual inspection, using a four score system: ++, +, (+), and − indicates strong, weak, very weak and no binding, respectively.

The constructs HGFP_CBD500-118 and HGFP_CBD118-500, in which both cell wall binding domains were directly fused to each other in both orientations, and attached to an N-terminal GFP domain, both showed weak binding to all strains of serovars 4, 5, and 6 strains tested. As this corresponds to the binding pattern of the mere CBD500, these results suggested that CBD118 is not functional in these constructs. On the other hand it showed that CBD500 does not need to be located at the C-terminus of a protein to retain functionality. Assuming that enhanced flexibility of both binding domains in a fusion construct might render CBD118 functional, we created the proteins HGFP_CBD500L118 and HGFP_CBD118L500, which include the linker peptide of PlyPSA separating the CBDs. Again, both constructs labelled all strains belonging to serovar 4, 5, and 6 strains, but additionally also four out of seven serovar 1/2 strains were tested. From fluorescence microscopy it was observed that the latter were predominantly marked at the poles and septa as observed for HGFP_CBD118. In contrast, both proteins decorated strains of serovars 4, 5, and 6 in even distribution over the cell surfaces like HGFP_CBD500. Thus, these double CBD constructs combined properties of both CBDs, although their binding ranges within serovars 1/2, 3, and “7” were narrower than that of HGFP_CBD118. Introduction of a short linker not only enabled CBD118 to access its ligands, but it also enhanced binding of CBD500 in C-terminal position. The fusion protein HGFP_CBD118L500 displayed equally strong decoration of most of the serovar 4, 5, and 6 cells as HGFP_CBD500. In addition, two fusion constructs were generated in which the GFP was placed in central position, whereas CBD500 and CBD118 were either N- or C-terminally located. In HCBD500_GFP_CBD118, CBD118 was directly attached to the C-terminus of the GFP, placing it in the same environment as in HGFP_CBD118. This protein was able to mark all serovar 1/2 strains tested, although the decoration was very weak. The construct HCBD118_GFP_CBD500, in which the CBDs were inversely oriented, strongly bound to most of the serovar 4, 5, and 6 strains, but only weakly labelled one serovar 1/2 strain. Again, CBD500 showed stronger binding when located at the C-terminus. However, it was demonstrated to be functional also in N-terminal position (HCBD500_GFP_CBD118).

TABLE 1
Binding of GFP-tagged CBDs and double CBD fusion proteins from different Listeria endolysins to Listeria cells from different
species and serovars.

| | | | | Binding of HGFP_CBD | | | | | | | | | | |
|---|---|---|---|---|---|---|---|---|---|---|---|---|---|---|
| | WLSC | | | | | | 500- | 118- | | | | | 500- | P35- |
| Species | code | Source | SV | 500 | 118 | P35 | 118 | 500 | 500L118 | 118L500 | 500G118 | 118G500 | P35 | 500 |
| L. monocytogenes | EGDe | J. Kreft | 1/2a | − | ++ | ++ | − | − | + | (+) | (+) | − | ++ | ++ |
| L. monocytogenes | 10403S | D. Portnoy | 1/2a | − | ++ | ++ | − | − | − | − | (+) | − | ++ | + |
| L. monocytogenes | 1442 | Food | 1/2a | − | ++ | − | − | − | + | + | (+) | + | − | − |
| L. monocytogenes | 1066 | SLCC 8800 | 1/2b | − | ++ | ++ | − | − | − | − | (+) | − | ++ | ++ |
| L. monocytogenes | 1001 | ATCC 19112 | 1/2c | − | ++ | ++ | − | − | − | − | (+) | − | ++ | ++ |
| L. seeligeri | 4007 | ATCC 35967 | 1/2b | − | ++ | ++ | − | − | + | + | (+) | − | ++ | ++ |
| L. welshimeri | 50149 | SLCC 5877 | 1/2b | − | ++ | + | − | − | + | + | (+) | − | (+) | (+) |
| L. monocytogenes | 1485 | soft cheese | 3a | − | + | + | − | − | − | − | − | − | ++ | ++ |
| L. monocytogenes | 1031 | SLCC1694 | 3b | − | + | ++ | − | − | − | − | − | − | ++ | ++ |
| L. monocytogenes | 1032 | SLCC 2479 | 3c | − | + | ++ | − | − | − | − | − | − | ++ | ++ |
| L. seeligeri | 40127 | SLCC 8604 | 3b | − | + | ++ | − | − | − | − | − | − | ++ | ++ |
| L. monocytogenes | 1034 | SLCC 2482 | “7” | − | + | − | − | − | − | − | − | − | − | − |
| L. monocytogenes | 1020 | ATCC 19114 | 4a | ++ | − | ++ | + | + | + | ++ | + | ++ | ++ | ++ |
| L. monocytogenes | 1042 | ATCC 23074 | 4b | ++ | − | − | + | + | + | ++ | + | ++ | ++ | ++ |
| L. monocytogenes | ScottA | J. Jay | 4b | ++ | − | − | + | + | + | ++ | + | ++ | ++ | ++ |
| L. monocytogenes | 1019 | ATCC 19116 | 4c | ++ | − | ++ | + | + | + | ++ | + | ++ | ++ | ++ |
| L. monocytogenes | 1033 | ATCC 19117 | 4d | ++ | − | + | + | + | + | ++ | + | ++ | ++ | ++ |
| L. monocytogenes | 1018 | ATCC 19118 | 4e | ++ | − | (+) | + | + | + | ++ | + | ++ | ++ | ++ |
| L. ivanovii | 3009 | SLCC 4769 | 5 | ++ | − | ++ | + | + | + | ++ | + | ++ | ++ | ++ |
| L. ivanovii | 3010 | ATCC 19119 | 5 | ++ | − | ++ | + | + | + | ++ | + | ++ | ++ | ++ |
| (ssp. Ivanovii) | | | | | | | | | | | | | | |
| L. ivanovii | 3060 | SLCC 3765 | 5 | ++ | − | − | + | + | + | + | (+) | + | + | + |
| (ssp. | | | | | | | | | | | | | | |
| Iondoniensis) | | | | | | | | | | | | | | |
| L. innocua | 2011 | ATCC 33090 | 6a | ++ | − | − | + | + | + | + | (+) | + | ++ | ++ |
| L. innocua | 2012 | ATCC 33091 | 6b | ++ | − | ++ | + | + | + | ++ | + | ++ | ++ | ++ |
| L. welshimeri | 50146 | SLCC 7622 | 6a | ++ | − | + | + | + | + | ++ | + | ++ | ++ | ++ |
| L. grayi | 6036 | ATCC 19120 | − | (+) | (+) | ++ | − | − | − | − | − | − | + | ++ |
| (ssp. Grayi) | | | | | | | | | | | | | | |
| L. grayi | 6037 | ATCC 25401 | − | (+) | (+) | ++ | − | − | − | − | − | − | ++ | ++ |
| (ssp. Murrayi) | | | | | | | | | | | | | | |

500G118 and 118G500 stand for HCBD500_GFP_118 and HCBD118_GFP_500, respectively.
“L” stands for a linker introduced between the shuffled CBDs.
++ strong,
+ weak,
(+) very weak,
− no binding;
WLSC: Weihenstephan Listeria Strain Collection;
SV: Listeria serovar

In a further approach, CBD118 in double CBD fusion constructs was replaced by CBDP35 (SEQ ID NO:59). The CBD of the endolysin of phage P35 strongly labelled most strains of serovars 1/2 and 3 as well as some strains of serovars 4, 5, and 6, binding in even distribution over the complete cell surface. The binding patterns of the newly constructed proteins HGFP_CBD500-P35 and HGFP_CBDP35-500 represented almost exact combinations of those of the single CBDs of Ply500 and PlyP35: They displayed strong binding to all strains which were either bound by CBD500 or by CBDP35 or by both, regardless of the location of the single CBDs within the fusions. These results proved that a combination of two cell wall binding domains from different peptidoglycan lysing enzymes can be fully functional in artificial fusion proteins, even when they are not in C-terminal position.

### Example 4

### Determination of Binding Affinity by Surface Plasmon Resonance Analysis (SPR)

Affinities of HGFP_CBD500 (SEQ ID NO:60) and HGFP_CBD500-500 to the cell wall of L. monocytogenes WSLC 1042 were determined by surface plasmon resonance analysis, using a BIAcore X instrument and C1 sensor chips (BIAcore, Uppsala, Sweden). The chip surface was activated with the amine coupling method and coated with HGFP-CBD500 molecules in both flow cells (70 μl of 0.5 mg/ml protein in 10 mM sodium acetate buffer, pH 5, at a flow rate of 5 μl/min). Heat inactivated WSLC 1042 cells in HBS buffer (10 mM HEPES, 150 mM NaCl, 3.4 mM EDTA, 0.005% Tween 20, pH 7.8) were then bound to the immobilized CBDs in flow cell Fc2 (3.0×10¹⁰ cells per ml; 15 μl at a flow rate of 3 μl/min). Finally, interactions between the immobilized cells and 3 different concentrations of both HGFP_CBD500 (50 nM, 100 nM, 200 nM) and HGFP_CBD500-500 (12.5 nM, 25 nM, 50 nM) in HBS buffer were measured (30 μl at 10 μl/min), Fc1 serving as reference cell. The association phase was measured for 3 min, the dissociation phase for 12 min. All steps were carried out at 25° C. Evaluation of kinetic data was performed with the BIAevaluation software, version 4.1 (BIAcore), employing a “1:1 binding with mass transfer” model. The equilibrium association constants obtained for three concentrations measured for each protein are given in table 2.

TABLE 2
Equilibrium affinity constants (K_{A}) of HGFP_CBD500
and HGFP_CBD500-500 binding to the cell wall of
Listeria monocytogenes WSLC 1042.

| HGFP_CBD500 | | HGFP_CBD500-500 | |
|---|---|---|---|
| Concentration (nM) | K_{A} (M⁻¹) | Concentration (nM) | K_{A} (M⁻¹) |
| 200 | 5.61 × 10⁸ | 50 | 1.00 × 10¹⁰ |
| 100 | 6.50 × 10⁸ | 25 | 5.61 × 10¹⁰ |
| 50 | 5.96 × 10⁸ | 12.5 | 2.19 × 10¹⁰ |
| mean | 6.02 × 10⁸ | mean | 2.93 × 10¹⁰ |

The construct HGFP_CBD500-500 comprising the artificial double CBD was shown to bind to the immobilized Listeria cells with an approximately 50 fold higher affinity compared to HGFP_CBD500 comprising the natural CBD of the endolysin of phage A500-ply500. Comparing sensograms of both the single and double CBD protein constructs, it was obvious that both constructs mainly differed in the dissociation phase. Once bound to the cell surface, HGFP_CBD500 detached much more rapidly than HGFP_CBD500-500, resulting in the higher overall affinity of the double CBD construct.

### Example 5

### Photometric Lysis Assays

The lytic activity of wild-type and chimaeric peptidoglycan lysing enzymes was determined by a photometric lysis assay. Substrate cells of Listeria monocytogenes strains WSLC 1001 (serovar 1/2 c) and WSLC 1042 (serovar 4 b) were prepared by growing the bacteria in TB medium until late log phase and freezing them in 50-fold concentration in PBS buffer (50 mM NaH₂PO₄, 120 mM NaCl, pH 8.0). The assay was carried out in a total volume of 1 ml, with cells diluted to an initial OD₆₀₀ of approximately 1.0 in PBS. All purified native endolysins and chimeric proteins to be compared were added to the cells in equimolar amounts in a volume of 20 μl, and the OD at 600 nm was measured at intervals of 15 s for maximum 10 minutes. Enzymatic concentrations used ranged from 30 to 152 μmol/ml. For negative control, 20 μl buffer were added to the cells. All assays were carried out in triplicate. Loessner et al. (2002, Mol. Microbiol. 44, 335-349) suggested ionic interaction as the molecular basis for binding of CBDs to their ligands in the cell wall. The CBD of endolysin ply500 showed optimum binding at a NaCl concentration of approximately 100 mM and decreasing binding capacity with increasing salt concentration. Based on that, the lytic activity of his-tagged wild-type ply500 (HPL500) and a construct according to the invention using a duplication of the naturally occurring CBD500 of ply500 H_EAD_CBD500-500 was compared under high salt conditions. The assays were carried out as described above, but using NaCl concentrations (between 1M and 2 M). Photometric curves were normalized and corrected by the data of the control assays (corrected value=value+(1−control value)). The resulting curves were fitted with the following sigmoid function, using the software SigmaPlot 9.0 (Systat Software, Inc.): f=y0+a/(1+exp(−(x−x0)/b))̂c. The steepest slope of the function was determined, which corresponds to the relative enzymatic activity. Surprisingly, at salt concentrations of 1 M NaCl or higher, the peptidoglycan lysing enzyme according to the invention H_EAD_CBD500-500 showed higher lytic activity than the naturally occurring enzyme ply500.

### Example 6

### Protein Expression and Purification of Enterococcus Endolysins

The Enterococcus endolysins Fab25VL, Fab20VL, Fab20K, and the peptidoglycan lysing enzyme according to the invention EADFab25_CBD25_CBD₂₀ were expressed in and isolated from E. coli HMS174 DE3. Protein expression was performed for 3 h at 37° C. after induction with 1 mM IPTG. The bacterial cell pellet was harvested by centrifugation (5000 rpm, 15 min, 4° C.), resuspended in 25 ml buffer A (25 mM Tris, pH 8.0, 500 mM NaCl, 20 mM imidazol, 0.1% Tween 20, 10% glycerol), and the cells disrupted in a microfluidizer. Bacterial cell debris was removed by centrifugation (12000 rpm, 5 min, 4° C.). The supernatant was submitted to an ammonium sulphate precipitation to 30% saturation. The precipitate was collected by centrifugation (12000 rpm, 5 min, 4° C.). The supernatant including the endolyins was applied to hydrophobic chromatography using a 5 ml phenylsepharose column (High Sub FF, Amersham). The column was washed with 10 volumes of buffer B (25 mM Tris, pH 7.0, 500 mM NaCl, 30% ammonium sulphate, 10% glycerol). The endolysins were eluted with 10 column volumes of buffer C (25 mM Tris, pH 7.0, 500 mM NaCl, 10% glycerol). Protein containing fractions were analyzed for endolysin on Coomassie stained SDS-gels. Endolysin containing fractions were pooled and analyzed for lysis activity in plate lysis assays according to example 7.

### Example 7

### Plate Lysis Assay to Test the Lysis Activity and Host Range of Peptidoglycan Lysing Proteins Against Enterococcus Bacteria

A variety of Enterococcus bacteria from the medically relevant species Enterococcus faecium and Enterococcus faecalis were grown over night at 37° C. in precultures of 3 ml BHI medium. For each strain, 2 ml of the preculture was inoculated into 25 ml fresh medium and incubated up to an OD_{600 nm} of around 1. Bacterial cells were harvested by centrifugation at 4500 rpm for 15 min at 4° C. The cell pellet was resuspended in 500 μl BHI medium. For the test of lysis activity against heat inactivated cells (table 3), the cells were incubated at 85° C. for 45 min and collected by centrifugation at 1400 rpm. The cell pellet was resuspended in 10 ml LB top agar, and the top agar poured onto LB plates. For the test of lysis activity against living cells (table 4), Enterococcus precultures were grown in 1 ml BHI medium over night, the preculture mixed with 10 ml BHI top agar, poured onto LB plates, and incubated for 2 h at 30° C. The Enterococcus endolysins Fab25VL, Fab20K, an equimolar combination of the endolysins Fab25VL and Fab20K, and the artificial enzyme EADFab25_CBD25_CBD₂₀ according to the invention were used. 5 μl peptidoglycan lysing protein solution each were pipetted in spots onto the bacterial lawn immersed in the top agar. The appearance of lysis zones around the spotted protein solutions was analysed after 18 h of incubation of the plates at 30° C. using a four score system: +++, ++, +, and − indicates strong, medium, weak, weak and no lysis, respectively.

TABLE 3
Plate lysis assay using peptidoglycan lysing enzymes against heat inactivated Enterococcus cells

| | Strain | | | | 1:1 | |
|---|---|---|---|---|---|---|
| Species | (ProCC) | Source | Fab25VL | Fab20K | 25VL:20K | EADFab25_CBD25_CBD20 |
| E. faecium | 880 | Profos | +++ | − | +++ | +++ |
| E. faecium | 1177 | University | +++ | − | +++ | +++ |
| | | hospital | | | | |
| E. faecium | S1506 | ATCC | +++ | − | +++ | +++ |
| | | 20477 | | | | |
| E. faecium | S1553 | DSMZ | +++ | − | +++ | +++ |
| | | 2146 | | | | |
| E. faecium | S1563 | University | +++ | − | +++ | +++ |
| | | hospital | | | | |
| E. faecium | S1564 | University | +++ | − | +++ | +++ |
| | | hospital | | | | |
| E. faecium | S1565 | University | +++ | − | +++ | +++ |
| | | hospital | | | | |
| E. faecium | S1568 | University | +++ | − | +++ | +++ |
| | | hospital | | | | |
| E. faecium | S1570 | University | +++ | − | +++ | +++ |
| | | hospital | | | | |
| E. faecium | S1634 | Robert | +++ | − | +++ | +++ |
| | | Koch | | | | |
| | | Institute | | | | |
| E. faecalis | 17 | Prof. | ++ | +++ | +++ | +++ |
| | | Stetter | | | | |
| E. faecalis | 1176 | ATCC | − | +++ | +++ | +++ |
| | | 19433 | | | | |
| E. faecalis | S1505 | University | ++ | +++ | +++ | +++ |
| | | hospital | | | | |
| E. faecalis | S1507 | University | ++ | +++ | +++ | +++ |
| | | hospital | | | | |
| E. faecalis | S1552 | DSMZ | − | +++ | +++ | +++ |
| | | 2570 | | | | |
| E. faecalis | S1566 | University | +++ | +++ | +++ | +++ |
| | | hospital | | | | |
| E. faecalis | S1567 | University | ++ | +++ | +++ | +++ |
| | | hospital | | | | |
| E. faecalis | S1569 | University | +++ | +++ | +++ | +++ |
| | | hospital | | | | |
| E. faecalis | S1571 | University | ++ | +++ | +++ | +++ |
| | | hospital | | | | |
| E. faecalis | S1578 | University | − | +++ | +++ | +++ |
| | | hospital | | | | |
| E. faecalis | S2465 | University | +++ | +++ | +++ | +++ |
| | | hospital | | | | |

TABLE 4
Plate lysis assay using peptidoglycan lysing enzymes against living Enterococcus cells

| | Strain | | | | 1:1 | |
|---|---|---|---|---|---|---|
| Species | (ProCC) | Source | Fab25VL | Fab20K | 25VL:20K | EADFab25_CBD25_CBD20 |
| E. faecium | 880 | Profos | +++ | − | ++ | ++ |
| E. faecium | 1177 | University | ++ | − | ++ | +++ |
| | | hospital | | | | |
| E. faecium | S1506 | ATCC | + | − | + | ++ |
| | | 20477 | | | | |
| E. faecium | S1553 | DSMZ | ++ | − | + | +++ |
| | | 2146 | | | | |
| E. faecium | S1563 | University | − | − | − | +++ |
| | | hospital | | | | |
| E. faecium | S1564 | University | ++ | − | ++ | +++ |
| | | hospital | | | | |
| E. faecium | S1565 | University | ++ | − | ++ | ++ |
| | | hospital | | | | |
| E. faecium | S1568 | University | ++ | − | ++ | ++ |
| | | hospital | | | | |
| E. faecium | S1570 | University | + | − | − | ++ |
| | | hospital | | | | |
| E. faecium | S1634 | Robert | ++ | − | ++ | +++ |
| | | Koch | | | | |
| | | Institute | | | | |
| E. faecalis | 17 | Prof. | + | − | − | +++ |
| | | Stetter | | | | |
| E. faecalis | 1176 | ATCC | − | − | − | + |
| | | 19433 | | | | |
| E. faecalis | S1505 | University | + | − | + | +++ |
| | | hospital | | | | |
| E. faecalis | S1507 | University | − | − | − | +++ |
| | | hospital | | | | |
| E. faecalis | S1552 | DSMZ | − | − | − | +++ |
| | | 2570 | | | | |
| E. faecalis | S1566 | University | + | − | + | + |
| | | hospital | | | | |
| E. faecalis | S1567 | University | + | − | + | +++ |
| | | hospital | | | | |
| E. faecalis | S1569 | University | + | − | − | +++ |
| | | hospital | | | | |
| E. faecalis | S1572 | University | + | − | − | +++ |
| | | hospital | | | | |
| E. faecalis | S1578 | University | − | − | − | +++ |
| | | hospital | | | | |

It turned out that using heat inactivated Enterococcus cells (table 3), the endolysin Fab20K lysed all E. faecalis strains with high efficiency, but no strains of E. faecium. Fab20 Endolysin seemed to be specific for E. faecalis strains. Fab25VL lysed all strains of E. faecium with high efficiency, but only two strains of E. faecalis. The other E. faecalis strains where lysed with medium efficiency or were not lysed at all. The naturally occurring endolysin Fab25VL therefore was not strictly specific to E. faecium, but lysed strains from this species more reliably than strains from the species E. faecalis. A 1:1 mixture of the two endolysins Fab25VL and Fab20K lysed all strains tested with high efficiency, but the same was achieved using only one enzyme according to the invention, namely EADFab25_CBD25_CBD₂₀ which combines the CBDs of Fab25VL and Fab20K, but has only the EAD of Fab25VL. Using only one enzyme instead of two facilitates enzyme production, reduces costs, and, minimizes immunological reactions in therapeutic applications. Performing the assay using living cells (table 4), the advantages using the enzyme according to the invention EADFab25_CBD25_CBD₂₀ were more pronounced. Whereas Fab25VL lysed mainly E. faecium cells under these assay conditions, and only some E. faecalis cells with low efficiency, the endolysin Fab20K did not work at all. None of the living cells were lysed at all, suggesting that maybe the cell surface receptors for endolysin binding or the substrate molecules for peptidoglycan lysis were not accessible in living cells. A combination of the two enzymes did not improve the situation, but cell lysis using EADFab25_CBD25_CBD₂₀ gave much better results. All strains tested were lysed at least with low efficiency, but most of the strains were lysed with high efficiency. This unexpected result shows that the artificial peptidoglycan lysing enzymes according to the invention can have favourable effects in specific uses even if a first characterization suggests a pure additive effect of the more than one CBD used in terms of the bacterial host range.

### Example 8

Determination of the Minimal Bactericidal Concentration (Mbc) of Peptidoglycan Lysing Enzymes Against Enterococci. Enterococcus faecalis strain 17 was grown over night at 37° C. in BHI medium. The preculture was diluted 1:10 into 25 ml fresh medium and incubated at 37° C. up to an OD_{600 nm} of around 1. Bacterial cells were harvested by centrifugation at 4500 rpm for 5 min at 4° C., and the cell pellet was resuspended in lysis buffer (PBS (2.25 mM NaH₂PO₃, 7.75 mM Na₂HPO₃, 150 mM NaCl) including, 2 mM CaCl₂, 10 mM BSA) to a concentration of 10⁵ cfu/ml. Protein solutions of Fab25VL and EADFab25_CBD25_CBD₂₀ (1 mg/ml) were serially diluted to 50 μg/ml, 5 μg/ml, 0.5 μg/ml, 0.05 μg/ml, 0.005 μg/ml, 0.0005 μg/ml, and 0.00005 μg/ml in lysis buffer. 450 μl cell suspension and 50 μl protein solution of each concentration were mixed and incubated for 1 h at 37° C. As a control, lysis buffer without protein added was incubated. 100 μl of 1:10 and 1:100 dilutions of the lysis samples were plated to LB agar plates, incubated for 1 day at 37° C. and counted for cells surviving the lysis by the peptidoglycan lysing enzymes. The MBC99.9%, for example, is defined as the lowest enzyme concentration at which the initial bacterial cell concentration is reduced by a factor of 1000. In this case, the cfu/ml of surviving cells had to be lower than 10².

It was observed that the MBC against Enterococcus faecalis strain 17 was lower using the peptidoglycan lysing enzyme according to the invention EADFab25_CBD25_CBD₂₀ than the naturally occurring enzyme Fab25 VL. The MBC99.9% was 0.05 μg/ml with EADFab25_CBD25_CBD₂₀ whereas it was 5 μg/ml using Fab25VL. This result suggests a higher binding affinity of EADFab25_CBD25_CBD₂₀ to the bacterial cells and/or an increased lysis activity. Using the polypeptide according to the invention a factor 100 less protein has to be used in order to kill the pathogenic bacteria.

1. A recombinant polypeptide having the activity of binding and lysing of-bacteria, comprising at least one enzymatically active domain and at least two bacterial cell binding domains.

2. A recombinant polypeptide having the activity of binding bacteria, comprising at least two bacterial cell binding domains.

3. The recombinant polypeptide according to claim 1, comprising two enzymatically active domains.

4. The recombinant polypeptide according to claim 1, wherein the enzymatically active domain(s) and/or bacterial cell binding domains are derived from two different peptidoglycan lysing enzymes.

5. The recombinant polypeptide according to claim 1, wherein the enzymatically active domain(s) is/are selected from the group consisting of Amidase_{—}5 (bacteriophage peptidoglycan hydrolase, pfam05382), Amidase_{—}2 (N-acetylmuramoyl-L-alanine amidase, pfam01510), Amidase_{—}3 (N-acetylmuramoyl-L-alanine amidase, pfam01520), Transgly (transglycosylase, pfam00912), Peptidase_M23 (peptidase family M23, pfam01551), endolysin_autolysin (CD00737), Hydrolase_{—}2 (cell wall hydrolase, pfam07486), CHAP (amidase, pfam05257), Transglycosylase (transglycosylase like domain, pfam06737), MtlB (membrane-bound lytic murein transglycosylase B, COG2951), MtlA (membrane-bound lytic murein transglycosylase A, COG2821), MtlE (membrane-bound lytic murein transglycosylase E, COG0741), bacteriophagelambdalysozyme (lysis of the bond between N-acetylmuramic acid and N-acetylglucosamine, CD00736), Peptidase_M74 (penicillin-insensitive murein endopeptidase, pfam03411), SLT (transglycosylase SLT, pfam01464), Lys (C-type lysozyme/alpha-lactalbumin family, pfam00062), COG5632 (N-acetylmuramoyl-L-alanine amidase, COG5632), MepA (murein endopeptidase, COG3770), COG1215 (glycosyltransferase, COG1215), AmiC (N-acetylmuramoyl-L-alanine amidase, COG0860), Spr (cell wall-associated hydrolase, COG0791), bacteriophage_T4-like_lysozyme (lysis of the bond between N-acetylmuramic acid and N-acetylglucosamine, cd00735), LT_GEWL (lytic transglycosylase (LT) and goose egg white lysozyme (GEWL) domain, cd00254), peptidase_S66 (LD-carboxypeptidase, pfam02016), Glyco_hydro_{—}70 (glycosyl hydrolase family 70, pfam02324), Glyco_hydro_{—}25 (glycosyl hydrolase familiy 25), VanY (D-alanyl-D-alanine carboxypeptidase, pfam02557), and LYZ2 (lysozyme subfamily 2, smart 00047).

6. The recombinant polypeptide according to claim 1, wherein the bacterial cell bindings domains are selected from the group consisting of SH3_{—}5 (bacterial SH3 domain, pfam08460), SH3_{—}4 (bacterial SH3 domain, pfam06347), SH3_{—}3 (bacterial SH3 domain, pfam08239), SH3b (bacterial SH3 domain homologue, smart00287), LysM (LysM domain found in a variety of enzymes involved in cell wall degradation, pfam01476 and cd00118), PG_binding_{—}1 (putative peptidoglycan binding domain, pfam01471), PG_binding_{—}2 (putative peptidoglycan binding domain, pfam08823), MtlA (peptidoglycan binding domain from murein degrading transglycosylase, pfam03462), Cpl-7 (C-terminal domain of Cpl-7 lysozyme, pfam08230), CW_binding_{—}1 (putative cell wall binding repeat, pfam01473), LytB (putative cell wall-binding domain, COG2247), and LytE (LysM repeat, COG1388).

7. The recombinant polypeptide according to claim 1, wherein said domains are in the range of about 15 to about 250 amino acid residues long, particular in the range of about 20 to about 200 amino acid residues long and more particular about 15 to about 40 amino acid residues long.

8. The recombinant polypeptide according to claim 1, wherein the enzymatically active domain(s) and/or the bacterial cell binding domains are derived from wild-type peptidoglycan lysing enzymes selected from the group consisting of Ply500, Ply511, Ply118, Ply100, PlyP40, Ply3626, phiLM4 endolysin, PlyCD119, PlyPSAa, Ply21, PlyBA, Ply12, PlyP35, PlyPH, PlyL, PlyB, phi11 endolysin, phi MR11 endolysin, phi12 endolysin, S. aureus phage PVL amidase, plypitti26, ΦSA2usa endolysin, endolysin of Staphylococcus warneri M phage ΦWMY PlyGBS, B30 endolysin, Cpl-1, Cpl-7, Cpl-9, PlyG, PlyC, pal amidase, Fab25, Fab20, endolysins from the Enterococcus faecalis V583 prophage, lysostaphin, phage PL-1 amidase, S. capitis ALE-1 endopeptidase, mutanolysin (N-acetylmuramidase of Streptomyces globisporus ATCC 21553), enterolysin A (cell wall degrading bacteriocin from Enterococcus faecalis LMG 2333), LysK, LytM, Ami autolysin from L. monocytogenes, endolysins of the Pseudomonas aeruginosa phages ΦKZ and EL, T4 lysozyme, gp61 muramidase, and STM0016 muramidase.

9. The recombinant polypeptide according to claim 8, wherein the enzymatically active domain derived from PlyP40 is encoded by an amino acid sequence according to SEQ ID NO: 103 and/or wherein the bacterial cell binding domain derived from PlyP40 is encoded by an amino acid sequence according to SEQ ID NO: 104.

10. A recombinant polypeptide comprising an amino acid sequence as set forth as depicted in SEQ ID NO: 7, 9, 13, 15, 17, 19, 21, 23, 31, 33, 35, 37, 39, 41, 43, 45, 47, 49, 51, 71, 73, 75, 77, 79, 81, 83, 85, 87, 89, 91, 93, 95, 97, 99, or 101.

11. A nucleic acid molecule comprising a nucleotide sequence encoding for a recombinant polypeptide comprising:
a recombinant polypeptide having the activity of binding and lysing of bacteria, comprising at least one enzymatically active domain and at least two bacterial cell binding domains; or
a recombinant polypeptide having the activity of binding bacteria, comprising at least two bacterial cell binding domains.

12. The nucleic acid molecule according to claim 11, wherein the nucleic acid molecule comprises a nucleotide sequence as depicted in SEQ ID NO: 8, 10, 14, 16, 18, 20, 22, 24, 32, 34, 36, 38, 40, 42, 44, 46, 48, 50, 52, 72, 74, 76, 78, 80, 82, 84, 86, 88, 90, 92, 94, 96, 98, 100, or 102.

13. A vector comprising a nucleic acid molecule according to claim 11.

14. A medicament comprising a polypeptide:
a recombinant polypeptide having the activity of binding and lysing of bacteria, comprising at least one enzymatically active domain and at least two bacterial cell binding domains; or
a recombinant polypeptide having the activity of binding bacteria, comprising at least two bacterial cell binding domains.

15. A method of disinfecting, decontaminating or protecting an environment, facility or surface comprising contacting said environment, facility or surface with a recombinant polypeptide according to:
a recombinant polypeptide having the activity of binding and lysing of bacteria, comprising at least one enzymatically active domain and at least two bacterial cell binding domains; or
a recombinant polypeptide having the activity of binding bacteria, comprising at least two bacterial cell binding domains.

16. A method of diagnosing contamination of a medicine, food, feed or environment comprising (a) contacting said medicine, food, feed or environment with a recombinant polypeptide:
a recombinant polypeptide having the activity of binding and lysing of bacteria, comprising at least one enzymatically active domain and at least two bacterial cell binding domains; or
a recombinant polypeptide having the activity binding bacteria, comprising least two bacterial cell binding domains,
and (b) detecting binding of said recombinant polypeptide to a contaminant in said medicine, food, feed or environment.

17. The recombinant polypeptide according to claim 2, wherein the bacterial cell binding domains are derived from two different peptidoglycan lysing enzymes.

18. The recombinant polypeptide according to claim 2, wherein said domains are in the range of about 15 to about 250 amino acid residues long, particular in the range of about 20 to about 200 amino acid residues long and more particular about 15 to about 40 amino acid residues long.

19. The method of claim 15, wherein said environment is a medical, public or private environment, wherein said facility is a food industry, animal feed or cosmetic facility, and surface is a surface susceptible to bacterially contamination.

20. A vector comprising a nucleic acid molecule according to claims 12.
